(19) United States
(12) Patent Application Publication
(10) Pub. No.: US 2022/0071731 A1
(43) Pub. Date: Mar. 10, 2022

(54) MEDICAL IMPLANTS WITH STRUCTURAL MEMBERS HAVING FEATURES FOR RETAINING RADIOPAQUE MARKERS

(71) Applicant: Vesper Medical, Inc, Wayne, PA (US)

(72) Inventors: William James Harrison, Signal Mtn., TN (US); Michae Andrew Longo, Glenmoore, PA (US); Christopher John Turek, West Chester, PA (US); Timothy W. O'Neil, King of Prussia, PA (US); Daniel Alexander, Huntingdon Valley, PA (US)

(21) Appl. No.: 17/467,762

(22) Filed: Sep. 7, 2021

Related U.S. Application Data
(60) Provisional application No. 63/076,184, filed on Sep. 9, 2020.

(51) Int. Cl. A61B 90/00; A61F 2/82

(52) U.S. Cl. CPC A61B 90/39; A61F 2/82; A61F 2230/0069; A61B 2090/3966; A61B 2090/3991; A61F 2250/0098

(57) ABSTRACT

A medical implant may include a structural member and a radiopaque marker. The structural member may include a first surface, a second surface disposed opposite the first surface, and an opening extending from the first surface to the second surface. The opening may be defined by a first partial-cylindrical surface defining a first central axis, and a second partial-cylindrical surface defining a second central axis that is angled relative to the first central axis. The radiopaque marker may be disposed within the opening.

### CROSS-REFERENCE TO RELATED APPLICATIONS

This application claims priority to and the benefit of U.S. Provisional Patent Application No. 63/076,184, filed on Sep. 9, 2020, the disclosure of which is expressly incorporated herein by reference in its entirety.

### FIELD OF THE DISCLOSURE

The present disclosure relates generally to medical implants and more particularly to medical implants with structural members having features for retaining radiopaque markers.

### BACKGROUND OF THE DISCLOSURE

Various types of medical implants may include one or more radiopaque markers to facilitate visualization of the implant relative to the anatomy of a subject during and/or after implantation. For example, stents, such as intravascular stents, often may include one or more radiopaque markers connected to one or more structural members of the stent. A radiopaque marker generally may be positioned and secured within an opening or recess of a structural member of a medical implant. The radiopaque marker often may be formed of a precious or other rare earth metal, while the structural member may be formed of a different metal. In some instances, the medical implant may be subjected to significant temperature variations during manufacturing and/or use of the implant, and the thermal expansion properties of the two dissimilar metals may allow the radiopaque marker to become dislodged from the structural member. In the case of vascular implants, such dislodging of the radiopaque marker undesirably may create an embolus in the subject's blood stream.

According to existing techniques, a radiopaque marker may be secured to a structural member of a medical implant by welding or by mechanically pressing the radiopaque marker such that the marker is deformed to assume a shape that corresponds to a predefined shape of an opening or recess of the structural member. Welding of the radiopaque marker to the structural member may produce material oxides or other negative side effects from the welding, which can be detrimental to the implant and/or harmful to the subject. Although mechanically pressing the radiopaque marker within the opening or recess of the structural member may avoid the issues caused by welding, such an approach may not produce a secure bond between the marker and the structural member. Mechanical pressing techniques generally may rely on an interference fit for retaining the radiopaque marker within the opening or recess of the structural member. As noted above, certain medical implants may be exposed to extreme temperatures or other environmental conditions, for example during manufacturing of the implant and/or loading of the implant onto or within a delivery system, which may be detrimental to the security of the connection between the radiopaque marker and the structural member.

A need therefore exists for improved medical implants with structural members having features for retaining radiopaque markers and related methods for fabricating such medical implants, which may overcome one or more of the above-mentioned limitations associated with existing techniques for securing a radiopaque marker to a structural member of an implant.

### SUMMARY OF THE DISCLOSURE

The present disclosure provides medical implants and related methods for fabricating medical implants. In one aspect, a medical implant is provided. In one embodiment, the medical implant may include a structural member and a radiopaque marker. The structural member may include a first surface, a second surface disposed opposite the first surface, and an opening extending from the first surface to the second surface. The opening may be defined by a first partial-cylindrical surface defining a first central axis, and a second partial-cylindrical surface defining a second central axis that is angled relative to the first central axis. The radiopaque marker may be disposed within the opening.

In some embodiments, the first surface and the second surface may be curved surfaces. In some embodiments, the first surface and the second surface may be partial-cylindrical surfaces defining a longitudinal axis of the medical implant. In some embodiments, the first surface and the second surface may be planar surfaces, and the second surface may extend parallel to the first surface. In some embodiments, the first surface may be an external surface of the medical implant, and the second surface may be an internal surface of the medical implant.

In some embodiments, the second central axis and the first central axis may define a first acute angle therebetween. In some embodiments, the opening may define a central axis that extends perpendicular to the first surface, the first central axis may be angled relative to the central axis of the opening, and the second central axis may be angled relative to the central axis of the opening. In some embodiments, the first central axis may be angled relative to the central axis of the opening in a first angular direction, and the second central axis may be angled relative to the central axis of the opening in a second angular direction that is opposite the first angular direction. In some embodiments, the first central axis and the central axis of the opening may define a second acute angle therebetween, and the second central axis and the central axis of the opening may define a third acute angle therebetween. In some embodiments, the second acute angle may be equal to the third acute angle.

In some embodiments, the central axis of the opening may extend perpendicular to a longitudinal axis of the medical implant. In some embodiments, the first central axis may be offset from the second central axis by a first distance. In some embodiments, the first partial-cylindrical surface may have a first radius of curvature, and the second partial-cylindrical surface may have a second radius of curvature that is equal to the first radius of curvature. In some embodiments, the first central axis may be offset from the second central axis by the first distance in a direction extending perpendicular to the central axis of the opening. In some embodiments, the first central axis may be offset from the central axis of the opening by a second distance in a first linear direction, and the second central axis may be offset from the central axis of the opening by a third distance in a second linear direction that is opposite the first linear direction. In some embodiments, the second distance may be equal to the third distance. In some embodiments, the first partial-cylindrical surface may extend from the first surface to the second surface, and the second partial-cylindrical surface may extend from the first surface to the second surface. In some embodiments, the radiopaque marker may fill the opening. In some embodiments, the medical implant may be a stent. In some embodiments, the opening may be an eyelet of the stent.

In another aspect, a medical implant is provided. In one embodiment, the medical implant may include a structural member and a radiopaque marker. The structural member may include a first surface, a second surface disposed opposite the first surface, and an opening extending from the first surface to the second surface. The opening may be defined by a first partial-cylindrical surface defining a first central axis, a second partial-cylindrical surface defining a second central axis that is angled relative to the first central axis, and a third partial-cylindrical surface defining a third central axis that is angled relative to the first central axis. The radiopaque marker may be disposed within the opening.

In some embodiments, the first surface and the second surface may be curved surfaces. In some embodiments, the first surface and the second surface may be partial-cylindrical surfaces defining a longitudinal axis of the medical implant. In some embodiments, the first surface and the second surface may be planar surfaces. In some embodiments, the second surface may extend parallel to the first surface. In some embodiments, the first surface may be an external surface of the medical implant, and the second surface may be an internal surface of the medical implant. In some embodiments, the first central axis may extend perpendicular to the first surface. In some embodiments, the first central axis may extend perpendicular to the second surface. In some embodiments, the first central axis may extend perpendicular to a longitudinal axis of the medical implant. In some embodiments, the first central axis may be coaxial with a central axis of the opening.

In some embodiments, the second central axis may be angled relative to the first central axis in a first angular direction, and the third central axis may be angled relative to the first central axis in a second angular direction that is opposite the first angular direction. In some embodiments, the second central axis and the first central axis may define a first acute angle therebetween, and the third central axis and the first central axis may define a second acute angle therebetween. In some embodiments, the first acute angle may be within a range from 1 degree to 45 degrees, and the second acute angle may be within a range from 1 degree to 45 degrees. In some embodiments, the first acute angle may be equal to the second acute angle. In some embodiments, the first acute angle may be 20 degrees, and the second acute angle may be 20 degrees. In some embodiments, the third central axis and the second central axis may define a third acute angle therebetween.

In some embodiments, the second central axis may be offset from the first central axis by a first distance, and the third central axis may be offset from the first central axis by a second distance. In some embodiments, the second central axis may be offset from the first central axis by the first distance in a first linear direction, and the third central axis may be offset from the first central axis by the second distance in a second linear direction that is opposite the first linear direction. In some embodiments, the first partial-cylindrical surface may have a first radius of curvature, the first distance may be within a range from 40% to 160% of the first radius of curvature, and the second distance may be within a range from 40% to 160% of the first radius of curvature. In some embodiments, the first distance may be equal to the second distance. In some embodiments, the first distance may be equal to the first radius of curvature, and the second distance may be equal to the first radius of curvature. In some embodiments, the second partial-cylindrical surface may have a second radius of curvature that is less than the first radius of curvature. In some embodiments, the second radius of curvature may be greater than the first distance. In some embodiments, the third partial-cylindrical surface may have a third radius of curvature that is less than the first radius of curvature. In some embodiments, the third radius of curvature may be greater than the second distance. In some embodiments, the third radius of curvature may be equal to the second radius of curvature.

In some embodiments, the first partial-cylindrical surface may have a first radius of curvature, the second partial-cylindrical surface may have a second radius of curvature that is different from the first radius of curvature, and the third partial-cylindrical surface may have a third radius of curvature that is different from the first radius of curvature. In some embodiments, the second radius of curvature may be less than the first radius of curvature, and the third radius of curvature may be less than the first radius of curvature. In some embodiments, the third radius of curvature may be equal to the second radius of curvature. In some embodiments, the first partial-cylindrical surface may extend from the first surface to the second surface. In some embodiments, the second partial-cylindrical surface may extend from the first surface to the second surface. In some embodiments, the third partial-cylindrical surface may extend from the first surface to the second surface.

In some embodiments, the opening may be further defined by a first edge disposed along an intersection between the second partial-cylindrical surface and the first partial-cylindrical surface, and a second edge disposed along an intersection between the third partial-cylindrical surface and the first partial-cylindrical surface. In some embodiments, the opening may be further defined by a fourth partial-cylindrical surface defining a fourth central axis. In some embodiments, the fourth central axis may be coaxial with the first central axis. In some embodiments, the fourth partial-cylindrical surface may extend from the first surface to the second surface. In some embodiments, the opening may be further defined by a third edge disposed along an intersection between the fourth partial-cylindrical surface and the second partial-cylindrical surface, and a fourth edge disposed along an intersection between the fourth partial-cylindrical surface and the third partial-cylindrical surface. In some embodiments, the radiopaque marker may fill the opening. In some embodiments, the medical implant may be a stent. In some embodiments, the opening may be an eyelet of the stent.

In another aspect, a method for fabricating a medical implant is provided. In one embodiment, the method may include forming an opening extending from a first surface to a second surface of a structural member of the medical implant, and securing a radiopaque marker within the opening. The second surface may be disposed opposite the first surface. The opening may be defined by a first partial-cylindrical surface defining a first central axis, a second partial-cylindrical surface defining a second central axis that is angled relative to the first central axis, and a third partial-cylindrical surface defining a third central axis that is angled relative to the first central axis.

In some embodiments, forming the opening may include forming a cylindrical surface extending from the first surface to the second surface, and the first partial-cylindrical surface may be a first portion of the cylindrical surface. In some embodiments, the opening may be further defined by a fourth partial-cylindrical surface, and the fourth partial-cylindrical surface may be a second portion of the cylindrical surface. In some embodiments, forming the opening further may include removing part of the cylindrical surface to form the second partial-cylindrical surface. In some embodiments, forming the opening further may include removing part of the cylindrical surface to form the third partial-cylindrical surface. In some embodiments, the second central axis may be angled relative to the first central axis in a first angular direction, and the third central axis may be angled relative to the first central axis in a second angular direction that is opposite the first angular direction. In some embodiments, the second central axis and the first central axis may define a first acute angle therebetween, and the third central axis and the first central axis may define a second acute angle therebetween. In some embodiments, the second central axis may be offset from the first central axis by a first distance in a first linear direction, and the third central axis may be offset from the first central axis by a second distance in a second linear direction that is opposite the first linear direction. In some embodiments, securing the radiopaque marker within the opening may include deforming material of the radiopaque marker from a first configuration to a second configuration within the opening. In some embodiments, the medical implant may be a stent, and the opening may be an eyelet of a stent.

In still another aspect, a medical implant is provided. In one embodiment, the medical implant may include a structural member and a radiopaque marker. The structural member may include a first surface, a second surface disposed opposite the first surface, and an opening extending from the first surface to the second surface. The opening may be defined by a first partial-cylindrical surface defining a first central axis, and a second partial-cylindrical surface defining a second central axis that is angled relative to the first central axis. The radiopaque marker may be disposed within the opening.

In some embodiments, the first surface and the second surface may be curved surfaces. In some embodiments, the first surface and the second surface may be partial-cylindrical surfaces defining a longitudinal axis of the medical implant. In some embodiments, the first surface and the second surface may be planar surfaces. In some embodiments, the second surface may extend parallel to the first surface. In some embodiments, the first surface may be an external surface of the medical implant, and the second surface may be an internal surface of the medical implant.

In some embodiments, the second central axis and the first central axis may define a first acute angle therebetween. In some embodiments, the first acute angle may be within a range from 1 degree to 89 degrees. In some embodiments, the first acute angle may be 20 degrees. In some embodiments, the opening may define a central axis that extends perpendicular to the first surface, the first central axis may be angled relative to the central axis of the opening, and the second central axis may be angled relative to the central axis of the opening. In some embodiments, the first central axis may be angled relative to the central axis of the opening in a first angular direction, and the second central axis may be angled relative to the central axis of the opening in a second angular direction that is opposite the first angular direction. In some embodiments, the first central axis and the central axis of the opening may define a second acute angle therebetween, and the second central axis and the central axis of the opening may define a third acute angle therebetween. In some embodiments, the second acute angle may be within a range from 1 degree to 45 degrees, and the third acute angle may be within a range from 1 degree to 45 degrees. In some embodiments, the second acute angle may be equal to the third acute angle. In some embodiments, the second acute angle may be 10 degrees, and the third acute angle may be 10 degrees. In some embodiments, the central axis of the opening may extend perpendicular to a longitudinal axis of the medical implant.

In some embodiments, the first central axis may be offset from the second central axis by a first distance. In some embodiments, the first partial-cylindrical surface may have a first radius of curvature, and the first distance may be within a range from 40% to 90% of the first radius of curvature. In some embodiments, the first distance may be 66% of the first radius of curvature. In some embodiments, the second partial-cylindrical surface may have a second radius of curvature that is equal to the first radius of curvature. In some embodiments, the first central axis may be offset from the second central axis by the first distance in a direction extending perpendicular to the central axis of the opening. In some embodiments, the first central axis may be offset from the central axis of the opening by a second distance, and the second central axis may be offset from the central axis of the opening by a third distance. In some embodiments, the first central axis may be offset from the central axis of the opening by the second distance in a first linear direction, and the second central axis may be offset from the central axis of the opening by the third distance in a second linear direction that is opposite the first linear direction. In some embodiments, the first partial-cylindrical surface may have a first radius of curvature, the second distance may be within a range from 20% to 45% of the first radius of curvature, and the third distance may be within a range from 20% to 45% of the first radius of curvature. In some embodiments, the second distance may be equal to the third distance. In some embodiments, the first partial-cylindrical surface may extend from the first surface to the second surface, and the second partial-cylindrical surface may extend from the first surface to the second surface. In some embodiments, the opening may be further defined by a first edge disposed along a first intersection between the second partial-cylindrical surface and the first partial-cylindrical surface, and a second edge disposed along a second intersection between the second partial-cylindrical surface and the first partial-cylindrical surface. In some embodiments, the radiopaque marker may fill the opening. In some embodiments, the medical implant may be a stent. In some embodiments, the opening may be an eyelet of the stent.

In yet another aspect, a method for fabricating a medical implant is provided. In one embodiment, the method may include forming an opening extending from a first surface to a second surface of a structural member of the medical implant, and securing a radiopaque marker within the opening. The second surface may be disposed opposite the first surface. The opening may be defined by a first partial-cylindrical surface defining a first central axis, and a second partial-cylindrical surface defining a second central axis that is angled relative to the first central axis.

In some embodiments, forming the opening may include forming a cylindrical surface extending from the first surface to the second surface, and the first partial-cylindrical surface may be a first portion of the cylindrical surface. In some embodiments, forming the opening further may include removing part of the cylindrical surface to form the second partial-cylindrical surface. In some embodiments, the first central axis and the second central axis may define a first acute angle therebetween. In some embodiments, the opening may define a central axis that extends perpendicular to the first surface, the first central axis may be angled relative to the central axis of the opening, and the second central axis may be angled relative to the central axis of the opening. In some embodiments, the first central axis may be angled relative to the central axis of the opening in a first angular direction, and the second central axis may be angled relative to the central axis of the opening in a second angular direction that is opposite the first angular direction. In some embodiments, the first central axis and the central axis of the opening may define a second acute angle therebetween, and the second central axis and the central axis of the opening may define a third acute angle therebetween. In some embodiments, the first central axis may be offset from the central axis of the opening by a first distance in a first linear direction, and the second central axis may be offset from the central axis of the opening by a second distance in a second linear direction that is opposite the first linear direction. In some embodiments, securing the radiopaque marker within the opening may include deforming material of the radiopaque marker from a first configuration to a second configuration within the opening. In some embodiments, the medical implant may be a stent, and the opening may be an eyelet of a stent.

In another aspect, a medical implant is provided. In one embodiment, the medical implant may include a structural member and a radiopaque marker. The structural member may include a first surface, a second surface disposed opposite the first surface, and an opening extending from the first surface to the second surface. The opening may be defined by a cylindrical surface, a first counterbore extending from the first surface to the cylindrical surface, and a second counterbore extending from the second surface to the cylindrical surface. The radiopaque marker may be disposed within the opening.

In some embodiments, the first surface and the second surface may be curved surfaces. In some embodiments, the first surface and the second surface may be partial-cylindrical surfaces defining a longitudinal axis of the medical implant, and a central axis of the opening may extend perpendicular to the longitudinal axis. In some embodiments, the first surface and the second surface may be planar surfaces, and the second surface may extend parallel to the first surface. In some embodiments, the first surface may be an external surface of the medical implant, and the second surface may be an internal surface of the medical implant. In some embodiments, the cylindrical surface may have a first diameter, the first counterbore may have a second diameter that is within a range of 105% to 130% of the first diameter, and the second counterbore may have a third diameter that is within a range of 105% to 130% of the first diameter. In some embodiments, the structural member may have a wall thickness between the first surface to the second surface, the first counterbore may have a first depth relative to the first surface that is within a range of 5% to 40% of the wall thickness, and the second counterbore may have a second depth relative to the second surface that is within a range of 5% to 40% of the wall thickness. In some embodiments, the radiopaque marker may fill the opening. In some embodiments, the medical implant may be a stent. In some embodiments, the opening may be an eyelet of the stent.

In still another aspect, a medical implant is provided. In one embodiment, the medical implant may include a structural member and a radiopaque marker. The structural member may include a first surface, a second surface disposed opposite the first surface, and an opening extending from the first surface to the second surface. The opening may be defined by a cylindrical surface, a first concave surface having an annular shape and extending from the first surface to the cylindrical surface, and a second concave surface having an annular shape and extending from the second surface to the cylindrical surface. The radiopaque marker may be disposed within the opening.

In some embodiments, the first surface and the second surface may be curved surfaces. In some embodiments, the first surface and the second surface may be partial-cylindrical surfaces defining a longitudinal axis of the medical implant, and a central axis of the opening may extend perpendicular to the longitudinal axis. In some embodiments, the first surface and the second surface may be planar surfaces, and the second surface may extend parallel to the first surface. In some embodiments, the first surface may be an external surface of the medical implant, and the second surface may be an internal surface of the medical implant. In some embodiments, the cylindrical surface may have a first diameter, the first concave surface may have a first minimum diameter that is equal to the first diameter and a first maximum diameter that is within a range of 105% to 130% of the first diameter, and the second concave surface may have a second minimum diameter that is equal to the first diameter and a second maximum diameter that is within a range of 105% to 130% of the first diameter. In some embodiments, the structural member may have a wall thickness between the first surface to the second surface, the first concave surface may have a first depth relative to the first surface that is within a range of 5% to 40% of the wall thickness, and the second concave surface may have a second depth relative to the second surface that is within a range of 5% to 40% of the wall thickness. In some embodiments, the radiopaque marker may fill the opening. In some embodiments, the medical implant may be a stent. In some embodiments, the opening may be an eyelet of the stent.

In yet another aspect, a medical implant is provided. In one embodiment, the medical implant may include a structural member and a radiopaque marker. The structural member may include a first surface, a second surface disposed opposite the first surface, and an opening extending from the first surface to the second surface. The opening may be defined by a cylindrical surface, a first convex surface having an annular shape and extending from the first surface to the cylindrical surface, and a second convex surface having an annular shape and extending from the second surface to the cylindrical surface. The radiopaque marker may be disposed within the opening.

In some embodiments, the first surface and the second surface may be curved surfaces. In some embodiments, the first surface and the second surface may be partial-cylindrical surfaces defining a longitudinal axis of the medical implant, and a central axis of the opening may extend perpendicular to the longitudinal axis. In some embodiments, the first surface and the second surface may be planar surfaces, and the second surface may extend parallel to the first surface. In some embodiments, the first surface may be an external surface of the medical implant, and the second surface may be an internal surface of the medical implant. In some embodiments, the cylindrical surface may have a first diameter, the first convex surface may have a first minimum diameter that is equal to the first diameter and a first maximum diameter that is within a range of 105% to 130% of the first diameter, and the second convex surface may have a second minimum diameter that is equal to the first diameter and a second maximum diameter that is within a range of 105% to 130% of the first diameter. In some embodiments, the structural member may have a wall thickness between the first surface to the second surface, the first convex surface may have a first depth relative to the first surface that is within a range of 5% to 40% of the wall thickness, and the second convex surface may have a second depth relative to the second surface that is within a range of 5% to 40% of the wall thickness. In some embodiments, the radiopaque marker may fill the opening. In some embodiments, the medical implant may be a stent. In some embodiments, the opening may be an eyelet of the stent.

In another aspect, a medical implant is provided. In one embodiment, the medical implant may include a structural member and a radiopaque marker. The structural member may include a first surface, a second surface disposed opposite the first surface, and an opening extending from the first surface to the second surface. The opening may be defined by a first cylindrical surface extending from the first surface toward the second surface, a second cylindrical surface extending from the second surface toward the first surface, and an annular groove disposed between the first cylindrical surface and the second cylindrical surface. The radiopaque marker may be disposed within the opening.

In some embodiments, the first surface and the second surface may be curved surfaces. In some embodiments, the first surface and the second surface may be partial-cylindrical surfaces defining a longitudinal axis of the medical implant, and a central axis of the opening may extend perpendicular to the longitudinal axis. In some embodiments, the first surface and the second surface may be planar surfaces, and the second surface may extend parallel to the first surface. In some embodiments, the first surface may be an external surface of the medical implant, and the second surface may be an internal surface of the medical implant. In some embodiments, the first cylindrical surface may have a first diameter, the second cylindrical surface may have a second diameter that is equal to the first diameter, and the annular groove may have a minimum diameter that is equal to the first diameter and a maximum diameter that is within a range of 105% to 130% of the first diameter. In some embodiments, the structural member may have a wall thickness between the first surface to the second surface, and the annular groove may have a thickness in the direction between the first surface and the second surface that is within a range of 30% to 70% of the wall thickness. In some embodiments, the radiopaque marker may fill the opening. In some embodiments, the medical implant may be a stent. In some embodiments, the opening may be an eyelet of the stent.

In still another aspect, a medical implant is provided. In one embodiment, the medical implant may include a structural member and a radiopaque marker. The structural member may include a first surface, a second surface disposed opposite the first surface, and an opening extending from the first surface to the second surface. The opening may be defined by a partial-cylindrical surface extending between the first surface and the second surface, and a helical groove extending between the first surface and the second surface. The radiopaque marker may be disposed within the opening.

In some embodiments, the first surface and the second surface may be curved surfaces. In some embodiments, the first surface and the second surface may be partial-cylindrical surfaces defining a longitudinal axis of the medical implant, and a central axis of the opening may extend perpendicular to the longitudinal axis. In some embodiments, the first surface and the second surface may be planar surfaces, and the second surface may extend parallel to the first surface. In some embodiments, the first surface may be an external surface of the medical implant, and the second surface may be an internal surface of the medical implant. In some embodiments, the partial-cylindrical surface may have a first diameter, and the helical groove may have a minimum diameter that is equal to the first diameter and a maximum diameter that is within a range of 105% to 130% of the first diameter. In some embodiments, the partial-cylindrical surface may extend from the first surface to the second surface, and the helical groove may extend from the first surface to the second surface. In some embodiments, the radiopaque marker may fill the opening. In some embodiments, the medical implant may be a stent. In some embodiments, the opening may be an eyelet of the stent.

In yet another aspect, a medical implant is provided. In one embodiment, the medical implant may include a structural member and a radiopaque marker. The structural member may include a first surface, a second surface disposed opposite the first surface, and an opening extending from the first surface to the second surface. The opening may be defined by a first cylindrical surface extending from the first surface toward the second surface, a second cylindrical surface extending from the second surface toward the first surface, and a circumferential array of recesses disposed around a central axis of the opening and between the first cylindrical surface and the second cylindrical surface. The radiopaque marker may be disposed within the opening.

In some embodiments, the first surface and the second surface may be curved surfaces. In some embodiments, the first surface and the second surface may be partial-cylindrical surfaces defining a longitudinal axis of the medical implant, and the central axis of the opening may extend perpendicular to the longitudinal axis. In some embodiments, the first surface and the second surface may be planar surfaces, and the second surface may extend parallel to the first surface. In some embodiments, the first surface may be an external surface of the medical implant, and the second surface may be an internal surface of the medical implant. In some embodiments, the first cylindrical surface may have a first diameter, the second cylindrical surface may have a second diameter that is equal to the first diameter, open ends of the recesses may define a minimum diameter that is equal to the first diameter, and closed ends of the recesses may define a maximum diameter that is within a range of 105% to 130% of the first diameter. In some embodiments, the structural member may have a wall thickness between the first surface to the second surface, and each of the recesses may have a thickness in the direction between the first surface and the second surface that is within a range of 10% to 50% of the wall thickness. In some embodiments, the radiopaque marker may fill the opening. In some embodiments, the medical implant may be a stent. In some embodiments, the opening may be an eyelet of the stent.

These and other aspects and improvements of the present disclosure will become apparent to one of ordinary skill in the art upon review of the following detailed description when taken in conjunction with the several drawings and the appended claims.

### BRIEF DESCRIPTION OF THE DRAWINGS

FIG. 1A is a top perspective view of a portion of an example medical implant in accordance with one or more embodiments of the disclosure, the medical implant including a structural member having an opening defined therein.

FIG. 1B is a top view of a portion of the medical implant of FIG. 1A.

FIG. 1C is a cross-sectional perspective view of a portion of the medical implant of FIG. 1A, with the cross-section taken along plane 1C-1C of FIG. 1B.

FIG. 1D is a cross-sectional perspective view of a portion of the medical implant of FIG. 1A, with the cross-section taken along plane 1D-1D of FIG. 1B.

FIG. 1E is a top view of central axes defined by the medical implant of FIG. 1A.

FIG. 1F is a front view of central axes defined by the medical implant of FIG. 1A.

FIG. 1G is a cross-sectional front view of a portion of the medical implant of FIG. 1A, with the cross-section taken along plane 1C-1C of FIG. 1B, showing the opening.

FIG. 1H is a cross-sectional front view of a portion of the medical implant of FIG. 1A, with the cross-section taken along plane 1C-1C of FIG. 1B, showing the opening and a radiopaque marker disposed within the opening.

FIG. 2A is a top perspective view of a portion of an example medical implant in accordance with one or more embodiments of the disclosure, the medical implant including a structural member having an opening defined therein.

FIG. 2B is a top view of a portion of the medical implant of FIG. 2A.

FIG. 2C is a cross-sectional perspective view of a portion of the medical implant of FIG. 2A, with the cross-section taken along plane 2C-2C of FIG. 2B.

FIG. 2D is a cross-sectional perspective view of a portion of the medical implant of FIG. 2A, with the cross-section taken along plane 2D-2D of FIG. 2B.

FIG. 2E is a top view of central axes defined by the medical implant of FIG. 2A.

FIG. 2F is a front view of central axes defined by the medical implant of FIG. 2A.

FIG. 2G is a cross-sectional front view of a portion of the medical implant of FIG. 2A, with the cross-section taken along plane 2C-2C of FIG. 2B, showing the opening.

FIG. 2H is a cross-sectional front view of a portion of the medical implant of FIG. 2A, with the cross-section taken along plane 2C-2C of FIG. 2B, showing the opening and a radiopaque marker disposed within the opening.

FIG. 3A is a top perspective view of a portion of an example medical implant in accordance with one or more embodiments of the disclosure, the medical implant including a structural member having an opening defined therein.

FIG. 3B is a top view of a portion of the medical implant of FIG. 3A.

FIG. 3C is a detailed cross-sectional front view of a portion of the medical implant of FIG. 3A, with the cross-section taken along plane 3C-3C of FIG. 3B, showing the opening.

FIG. 3D is a detailed cross-sectional front view of a portion of the medical implant of FIG. 3A, with the cross-section taken along plane 3C-3C of FIG. 3B, showing the opening and a radiopaque marker disposed within the opening.

FIG. 4A is a top perspective view of a portion of an example medical implant in accordance with one or more embodiments of the disclosure, the medical implant including a structural member having an opening defined therein.

FIG. 4B is a top view of a portion of the medical implant of FIG. 4A.

FIG. 4C is a front view of a portion of the medical implant of FIG. 4A.

FIG. 4D is a detailed cross-sectional front view of a portion of the medical implant of FIG. 4A, with the cross-section taken along plane 4D-4D of FIG. 4B, showing the opening.

FIG. 4E is a detailed cross-sectional front view of a portion of the medical implant of FIG. 4A, with the cross-section taken along plane 4D-4D of FIG. 4B, showing the opening and a radiopaque marker disposed within the opening.

FIG. 5A is a top perspective view of a portion of an example medical implant in accordance with one or more embodiments of the disclosure, the medical implant including a structural member having an opening defined therein.

FIG. 5B is a top view of a portion of the medical implant of FIG. 5A.

FIG. 5C is a front view of a portion of the medical implant of FIG. 5A.

FIG. 5D is a detailed cross-sectional front view of a portion of the medical implant of FIG. 5A, with the cross-section taken along plane 5D-5D of FIG. 5B, showing the opening.

FIG. 5E is a detailed cross-sectional front view of a portion of the medical implant of FIG. 5A, with the cross-section taken along plane 5D-5D of FIG. 5B, showing the opening and a radiopaque marker disposed within the opening.

FIG. 6A is a top perspective view of a portion of an example medical implant in accordance with one or more embodiments of the disclosure, the medical implant including a structural member having an opening defined therein.

FIG. 6B is a top view of a portion of the medical implant of FIG. 6A.

FIG. 6C is a front view of a portion of the medical implant of FIG. 6A.

FIG. 6D is a detailed cross-sectional front view of a portion of the medical implant of FIG. 6A, with the cross-section taken along plane 6D-6D of FIG. 6B, showing the opening.

FIG. 6E is a detailed cross-sectional front view of a portion of the medical implant of FIG. 6A, with the cross-section taken along plane 6D-6D of FIG. 6B, showing the opening and a radiopaque marker disposed within the opening.

FIG. 7A is a top perspective view of a portion of an example medical implant in accordance with one or more embodiments of the disclosure, the medical implant including a structural member having an opening defined therein.

FIG. 7B is a top view of a portion of the medical implant of FIG. 7A.

FIG. 7C is a side view of a portion of the medical implant of FIG. 7A.

FIG. 7D is a detailed cross-sectional front view of a portion of the medical implant of FIG. 7A, with the cross-section taken along plane 7D-7D of FIG. 7B, showing the opening.

FIG. 7E is a detailed cross-sectional front view of a portion of the medical implant of FIG. 7A, with the cross-section taken along plane 7D-7D of FIG. 7B, showing the opening and a radiopaque marker disposed within the opening.

FIG. 8A is a top perspective view of a portion of an example medical implant in accordance with one or more embodiments of the disclosure, the medical implant including a structural member having an opening defined therein.

FIG. 8B is a top view of a portion of the medical implant of FIG. 8A.

FIG. 8C is a detailed cross-sectional front view of a portion of the medical implant of FIG. 8A, with the cross-section taken along plane 8C-8C of FIG. 8B, showing the opening.

FIG. 8D is a detailed cross-sectional front view of a portion of the medical implant of FIG. 8A, with the cross-section taken along plane 8C-8C of FIG. 8B, showing the opening and a radiopaque marker disposed within the opening.

The detailed description is set forth with reference to the accompanying drawings. The drawings are provided for purposes of illustration only and merely depict example embodiments of the disclosure. The drawings are provided to facilitate understanding of the disclosure and shall not be deemed to limit the breadth, scope, or applicability of the disclosure. The use of the same reference numerals indicates similar, but not necessarily the same or identical components. Different reference numerals may be used to identify similar components. Various embodiments may utilize elements or components other than those illustrated in the drawings, and some elements and/or components may not be present in various embodiments. The use of singular terminology to describe a component or element may, depending on the context, encompass a plural number of such components or elements and vice versa.

### DETAILED DESCRIPTION OF THE DISCLOSURE

In the following description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional. In some instances, well known methods, procedures, and components have not been described in detail so as not to unnecessarily obscure aspects of the embodiments.

Overview

Embodiments of medical implants with structural members having features for retaining radiopaque markers (which also may be referred to as “radiopaque elements,” “radiopaque members,” “radiopaque marker inserts,” “radiopaque inserts,” or “marker inserts”) and related methods for fabricating such medical implants are provided herein. In some instances, the medical implants may be stents, such as intravascular stents, although the medical implants may be any type of implant having one or more radiopaque markers connected to one or more structural members of the implant. The radiopaque markers may be provided to facilitate visualization, via X-ray imaging and the like, of the implant relative to the anatomy of a subject during and/or after implantation. The radiopaque markers may be formed of a precious or other rare earth metal, such as gold and the like, while the structural members may be formed of a different metal.

The medical implants provided herein generally may include a structural member and a radiopaque marker connected to the structural member. The structural member may include a first surface, a second surface disposed opposite the first surface, and an opening extending from the first surface to the second surface and defining a central axis. The radiopaque marker may be disposed within the opening. The structural member may include one or more features defining the opening and configured to facilitate retention of the radiopaque marker. In other words, the one or more features of the structural member may cooperate with the radiopaque marker to retain the marker within the opening, inhibiting the marker from becoming disconnected from the structural member. As described herein, various geometries of such features and the openings defined thereby may be used. In some embodiments, the opening may be defined by two or more partial-cylindrical surfaces, with respective central axes defined by the partial-cylindrical surfaces being angled relative to one another and offset from one another. In some embodiments, the opening may be defined by a cylindrical surface and a pair of counterbores extending from respective ends of the cylindrical surface. In some embodiments, the opening may be defined by a cylindrical surface and a pair of concave surfaces extending from respective ends of the cylindrical surface. In some embodiments, the opening may be defined by a cylindrical surface and a pair of convex surfaces extending from respective ends of the cylindrical surface. In some embodiments, the opening may be defined by a pair of cylindrical surfaces and an annular groove disposed between the cylindrical surfaces. In some embodiments, the opening may be defined by a partial-cylindrical surface and a helical groove extending along the partial-cylindrical surface. In some embodiments, the opening may be defined by a pair of cylindrical surfaces and a circumferential array of recesses disposed between the cylindrical surfaces. Various combinations of such features may be used in other embodiments.

As discussed above, existing techniques for securing a radiopaque marker to a structural member of a medical implant may have certain limitations. In some instances, a radiopaque marker may be welded to a structural member of a medical implant. However, such welding may produce material oxides or other negative side effects from the welding, which can be detrimental to the implant and/or harmful to the subject. In some instances, a radiopaque marker may be mechanically pressed such that the marker is deformed to assume a shape that corresponds to a predefined shape of an opening of a structural member of a medical implant. For example, the opening may have a cylindrical or frustoconical shape, and the radiopaque marker may be placed within the opening and deformed from a spherical shape to a cylindrical or frustoconical shape to match the opening. In this manner, an interference fit may be provided for retaining the radiopaque marker within the opening. Although pressing and deforming the radiopaque marker may avoid the issues caused by welding, such an approach may not produce a secure bond between the marker and the structural member. Certain types of medical implants may be exposed to extreme temperatures or other environmental conditions, for example during manufacturing of the implant, loading of the implant onto or within a delivery system, and/or use of the implant. In some instances, such temperatures may be detrimental to the security of the connection between the radiopaque marker and the structural member. For example, the thermal expansion properties of the metal used for the radiopaque marker and the different metal used for the structural member may compromise the interference-fit connection, allowing the marker to become dislodged from the structural member. Such separation of the radiopaque marker from the structural member may be problematic for various types of medical implants, raising significant concerns with respect to the subject's safety. For example, in the case of vascular implants, a dislodged radiopaque marker undesirably may create an embolus in the subject's blood stream.

The medical implants and related methods provided herein advantageously may overcome one or more of the limitations associated with existing techniques for securing a radiopaque marker to a structural member of a medical implant. Notably, the disclosed medical implants do not rely on welds for securing a radiopaque marker to a structural member, and thus the implants avoid the welding-related problems mentioned above. As described herein, the medical implants include a structural member having an opening, and a radiopaque marker that is positioned and deformed within the opening to assume a shape corresponding to a shape of the opening. However, in contrast to existing techniques that utilize a conventional cylindrical or frustoconical opening, the implants provided herein utilize openings that are defined by one or more features configured to facilitate retention of the radiopaque marker. As described below, various geometries of the openings, as may be defined by one or more partial-cylindrical surfaces, cylindrical surfaces, counterbores, concave surfaces, convex surfaces, annular grooves, helical grooves, or any combination of such features, may be used. The resulting connection between the radiopaque marker and the structural member thus may provide enhanced retention of the marker, allowing the medical implant to withstand extreme temperatures or other environmental conditions without compromising the integrity of the connection.

Still other benefits and advantages of the medical implants and fabrication methods provided herein over existing technology for securing a radiopaque marker to a structural member of an implant will be appreciated by those of ordinary skill in the art from the following description and the appended drawings.

Defined Terms

As used herein, the term “defined by,” when referring to an opening being “defined by” one or more surfaces and/or edges, means that the one or more surfaces and/or edges define(s) at least a portion of the opening. In this manner, the term “defined by” is not exclusive, as use of the term leaves open the possibility that one or more additional surfaces and/or edges may define one or more additional portions of the opening. As used herein, the term “defined entirely by,” when referring to an opening being “defined entirely by” one or more surfaces and/or edges, means that the one or more surfaces and/or edges define(s) the entirety of the opening. In this manner, the term “defined entirely by” is exclusive with respect to additional surfaces or edges.

As used herein, the term “partial-cylindrical surface” means a curved surface that has a constant radius of curvature about a central axis but constitutes less than a complete cylindrical surface. As used herein, the term “central axis,” when referring to a partial-cylindrical surface defining a “central axis,” means the axis from which the constant radius of curvature of the partial-cylindrical surface extends.

As used herein, the term “substantially perpendicular,” when referring to a relationship between two features, means that the features are perpendicular to one another or within 5 degrees of being perpendicular to one another.

As used herein, the term “substantially parallel,” when referring to a relationship between two features, means that the features are parallel to one another or within 5 degrees of being parallel to one another.

As used herein, a recitation of a numerical range is inclusive of the bounds used to define the range. For example, a “range from X to Y” is inclusive of “X” and “Y.”

### Example Embodiments of Medical Implants

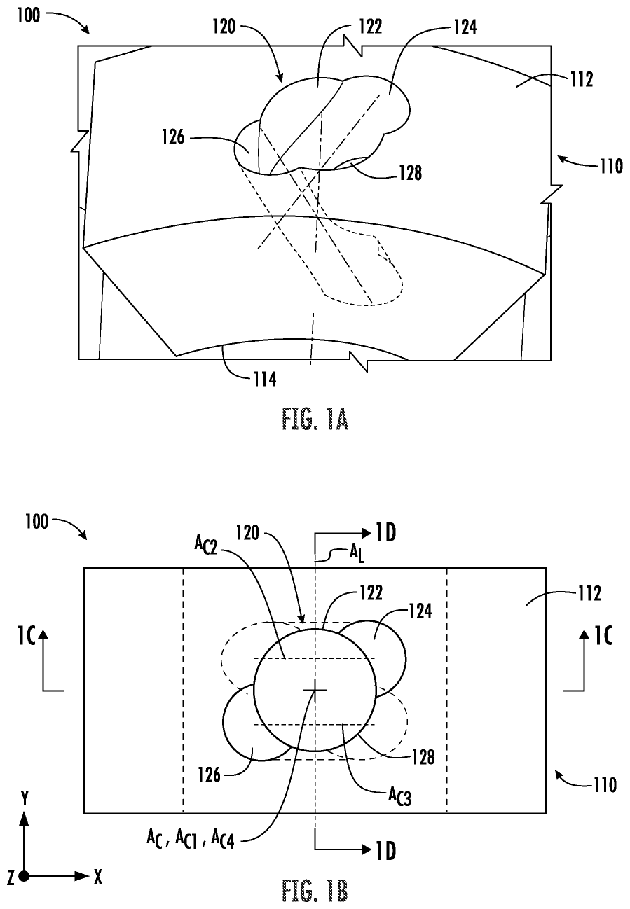

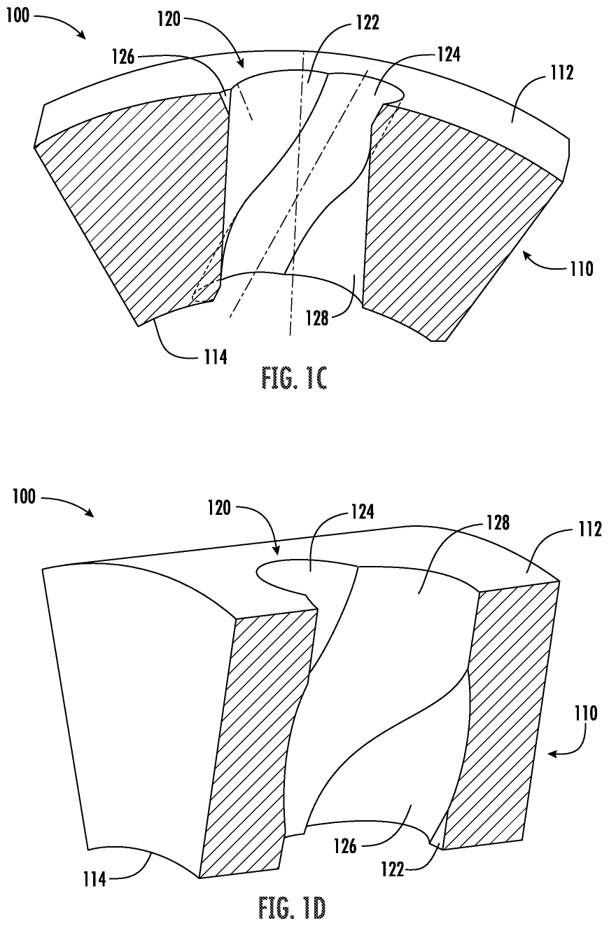

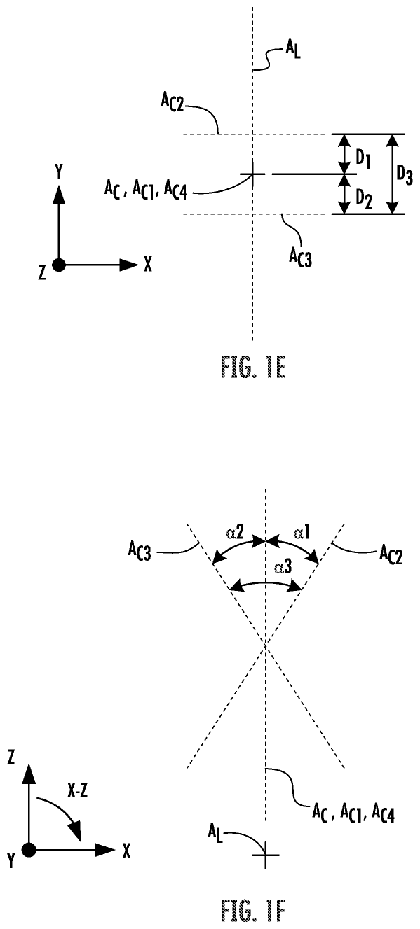

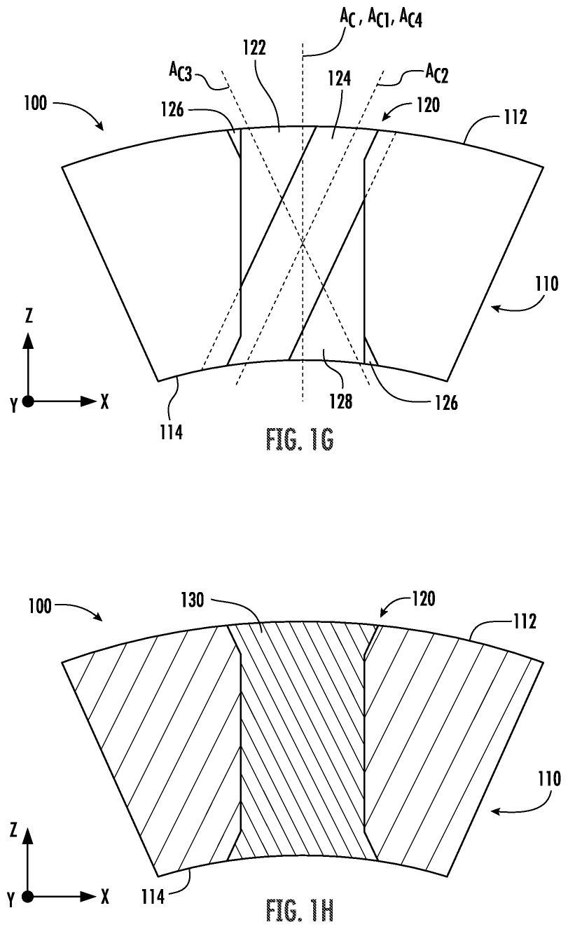

Referring now to FIGS. 1A-1H, a portion of an example medical implant 100 (which also may be referred to as a “medical device” or simply an “implant”) is depicted. Respective X, Y, and Z axes are shown in the figures to facilitate description of certain features of the illustrated embodiment and relationships therebetween. In some embodiments, the medical implant 100 may be a stent, such as an intravascular stent, although various other configurations of the implant 100 may be used in other embodiments. In some embodiments, the medical implant 100 may be formed as an elongated structure having a longitudinal axis A_{L} (extending in the Y direction). For example, when the implant 100 is a stent, the longitudinal axis A_{L} may be the axis with respect to which the stent radially expands. The implant 100 may include a structural member 110 (which also may be referred to as a “base member” or a “base”) and a radiopaque marker 130 (which also may be referred to as a “radiopaque element,” a “radiopaque member,” a “radiopaque marker insert,” a “radiopaque insert,” or a “marker insert”) that is fixedly connected to the structural member 110. In other words, after assembly, the radiopaque marker 130 is not intended to be removed from the structural member 110. In some embodiments, the structural member 110 may be a beam of the medical implant 100. For example, when the implant 100 is a stent, the structural member 110 may be a beam of the stent, such as a strut of the stent.

As shown, the structural member 110 may have a first surface 112, a second surface 114 disposed opposite the first surface 112 (in the Z direction), and an opening 120 (which also may be referred to as a “hole,” a “through-hole,” or an “aperture”) that extends from the first surface 112 to the second surface 114 (in the Z direction). The structural member 110 may have a wall thickness between the first surface 112 and the second surface 114 (in the Z direction). In some embodiments, as shown, the structural member 110 may have a constant wall thickness between the first surface 112 and the second surface 114. In some embodiments, as shown, the first surface 112 and the second surface 114 may be curved surfaces. For such embodiments, references herein to a feature (e.g., an axis, a surface, etc.) extending “perpendicular” to the first surface 112 and/or the second surface 114 mean that the feature extends perpendicular to a tangent of the curved surface at a location where the feature intersects the first surface 112 and/or the second surface 114. In some embodiments, as shown, the first surface 112 and the second surface 114 may be partial-cylindrical surfaces that define the longitudinal axis A_{L} of the implant 100. In some embodiments, the first surface 112 and the second surface 114 may be planar surfaces. In some embodiments, the second surface 114 may extend parallel, or substantially parallel, to the first surface 112. In some embodiments, the first surface 112 may be an external surface of the implant 100, and the second surface 114 may be an internal surface of the implant 100. For example, when the implant 100 is a stent, the first surface 112 may be a radially outer surface of the implant 100, and the second surface 114 may be a radially inner surface of the implant 100. The opening 120 may have a central axis A_{C} extending (in the Z direction) through a center of the opening 120. In some embodiments, as shown, the central axis A_{C} may extend perpendicular, or substantially perpendicular, to the longitudinal axis A_{L}. In some embodiments, the central axis A_{C} may extend perpendicular, or substantially perpendicular, to one or both of the first surface 112 and the second surface 114. In some embodiments, when the implant 100 is a stent, the opening 120 may be an eyelet of the stent. In some embodiments, as shown in FIG. 1H, the radiopaque marker 130 may fill the opening 120. In some embodiments, the radiopaque marker 130 may be flush with each of the first surface 112 and the second surface 114, as shown.

The opening 120 may be defined by a plurality of surfaces and/or edges. According to the illustrated embodiment, the opening 120 may be defined by a first partial-cylindrical surface 122 (which also may be referred to as a “first curved surface”), a second partial-cylindrical surface 124 (which also may be referred to as a “second curved surface”), a third partial-cylindrical surface 126 (which also may be referred to as a “third curved surface”), and a fourth partial-cylindrical surface 128 (which also may be referred to as a “fourth curved surface”). As shown, the first partial-cylindrical surface 122 may define a first central axis A_{C1}, the second partial-cylindrical surface 124 may define a second central axis A_{C2}, the third partial-cylindrical surface 126 may define a third central axis A_{C3}, and the fourth partial-cylindrical surface 128 may define a fourth central axis A_{C4}. In some embodiments, as shown, the first central axis A_{C1} may be coaxial with the central axis A_{C} of the opening 120. In some embodiments, the first central axis A_{C1} may extend perpendicular, or substantially perpendicular, to the longitudinal axis A_{L}. In some embodiments, the first central axis A_{C1} may extend perpendicular, or substantially perpendicular, to one or both of the first surface 112 and the second surface 114. In some embodiments, as shown, the fourth central axis A_{C4} may be coaxial with the central axis A_{C} of the opening 120 and the first central axis A_{C1}.

As shown in FIG. 1F, the second central axis A_{C2} may be angled (in the X-Z direction) relative to the first central axis A_{C1}, and the third central axis A_{C3} may be angled (in the X-Z direction) relative to the first central axis A_{C1}. Further, the third central axis A_{C3} may be angled (in the X-Z direction) relative to the second central axis A_{C2}. As shown, the second central axis A_{C2} may be angled relative to the first central axis A_{C1} in a first angular direction (the “+” X-Z direction in the illustrated embodiment), and the third central axis A_{C3} may be angled relative to the first central axis A_{C1} in a second angular direction (the “−”X-Z direction in the illustrated embodiment) that is opposite the first angular direction. In some embodiments, the second central axis A_{C2} and the first central axis A_{C1} may define a first acute angle α1 therebetween, and the third central axis A_{C3} and the first central axis A_{C1} may define a second acute angle α2 therebetween. In some embodiments, the first acute angle α1 may be within a range from 1 degree to 45 degrees, and the second acute angle α2 may be within a range from 1 degree to 45 degrees. In some embodiments, the first acute angle α1 may be equal to the second acute angle α2. In some embodiments, the first acute angle α1 may be 20 degrees, and the second acute angle α1 may be 20 degrees. In some embodiments, the third central axis A_{C3} and the second central axis A_{C2} may define a third acute angle α3 therebetween, with the third acute angle α3 being equal to a sum of the first acute angle α1 and the second acute angle α2.

As shown, each of the partial-cylindrical surfaces 122, 124, 126, 128 may have a constant radius of curvature. The first partial-cylindrical surface 122 may have a first radius of curvature, the second partial-cylindrical surface 124 may have a second radius of curvature, the third partial-cylindrical surface 126 may have a third radius of curvature, and the fourth partial-cylindrical surface 128 may have a fourth radius of curvature. In some embodiments, the second radius of curvature may be different from the first radius of curvature, and the third radius of curvature may be different from the first radius of curvature. In some embodiments, as shown, the second radius of curvature may be less than the first radius of curvature, and the third radius of curvature may be less than the first radius of curvature. In some embodiments, as shown, the second radius of curvature may be equal to the third radius of curvature. In some embodiments, as shown, the fourth radius of curvature may be equal to the first radius of curvature. In some embodiments, the first partial-cylindrical surface 122 may extend from the first surface 112 to the second surface 114. In some embodiments, the second partial-cylindrical surface 124 may extend from the first surface 112 to the second surface 114. In some embodiments, the third partial-cylindrical surface 126 may extend from the first surface 112 to the second surface 114. In some embodiments, the fourth partial-cylindrical surface 128 may extend from the first surface 112 to the second surface 114.

As shown in FIG. 1E, the second central axis A_{C2} may be offset (in the Y direction) from the first central axis A_{C1} by a first distance D₁, and the third central axis A_{C3} may be offset (in the Y direction) from the first central axis A_{C1} by a second distance D₂. In some embodiments, the second central axis A_{C2} may be offset from the first central axis A_{C1} by the first distance D₁ in a first linear direction (the “+” Y direction in the illustrated embodiment), and the third central axis A_{C3} may be offset from the first central axis A_{C1} by the second distance D₂ in a second linear direction (the “−” Y direction in the illustrated embodiment) that is opposite the first linear direction. In some embodiments, the first distance D₁ may be within a range from 40% to 160% of the first radius of curvature, and the second distance D₂ may be within a range from 40% to 160% of the first radius of curvature. In some embodiments, the first distance D₁ may be equal to the second distance D₂. In some embodiments, the first distance D₁ may be equal to the first radius of curvature, and the second distance D₂ may be equal to the first radius of curvature. In some embodiments, the second radius of curvature may be greater than the first distance D₁. In some embodiments, the third radius of curvature may be greater than the second distance D₂. In some embodiments, the third central axis A_{C3} may be offset (in the Y direction) from the second central axis A_{C2} by a third distance D₃, with the third distance D₃ being equal to a sum of the first distance D₁ and the second distance D₂.

In some embodiments, the opening 120 may be further defined by edges formed along intersections between respective pairs of the partial-cylindrical surfaces 122, 124, 126, 128. As shown, the opening 120 may be further defined by a first edge disposed along an intersection between the second partial-cylindrical surface 124 and the first partial-cylindrical surface 122, a second edge disposed along an intersection between the third partial-cylindrical surface 126 and the first partial-cylindrical surface 122, a third edge disposed along an intersection between the fourth partial-cylindrical surface 128 and the second partial-cylindrical surface 124, and a fourth edge disposed along an intersection between the fourth partial-cylindrical surface 128 and the third partial-cylindrical surface 126. As shown, each of the edges may have a contoured shape corresponding to the angular and offset relationships of the central axes A_{C1}, A_{C2}, A_{C3}, A_{C4} and the radii of curvature of the partial-cylindrical surfaces 122, 124, 126, 128. In some embodiments, the opening 120 may be defined entirely by the first partial-cylindrical surface 122, the second partial-cylindrical surface 124, the third partial-cylindrical surface 126, the fourth partial-cylindrical surface 128, the first edge, the second edge, the third edge, and the fourth edge.

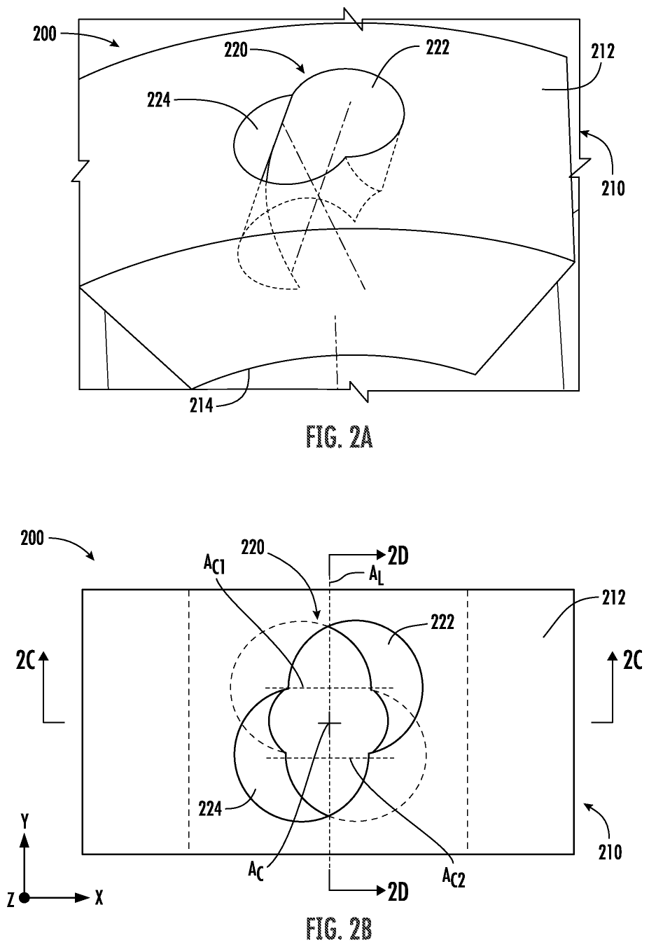

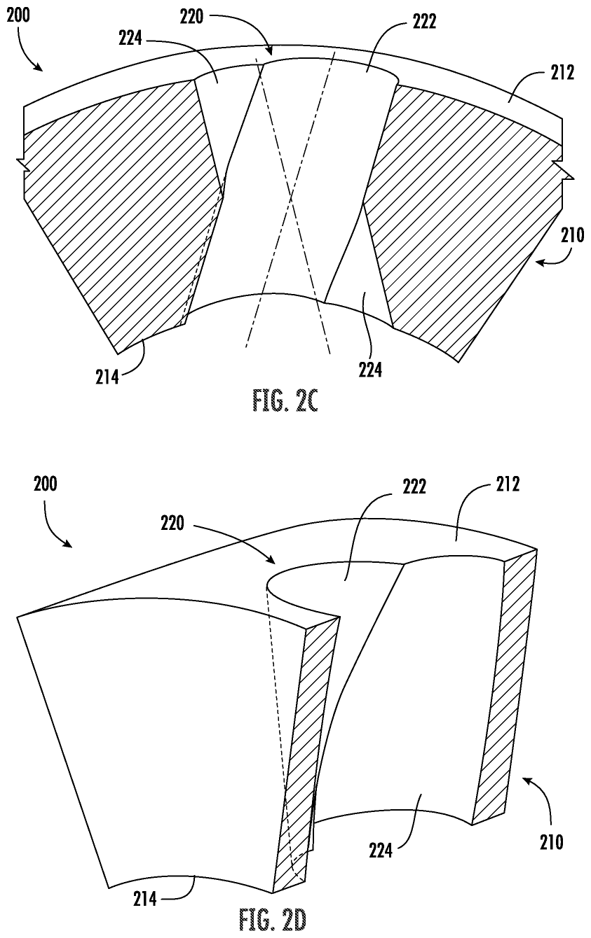

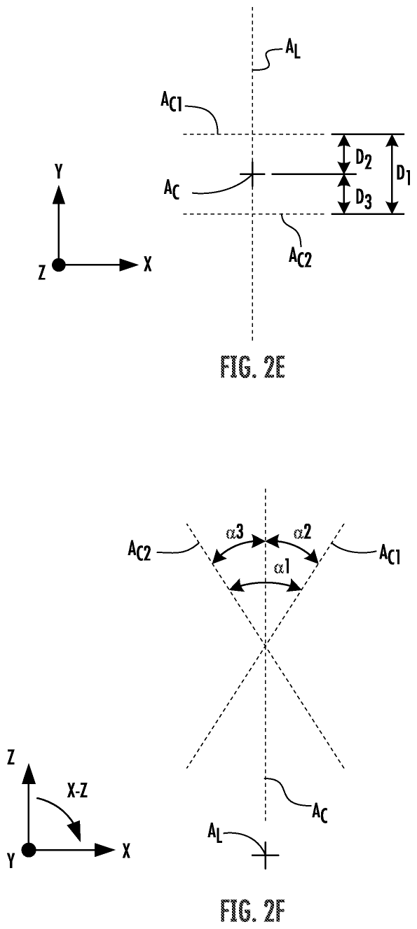

FIGS. 2A-2H depict a portion of another example medical implant 200 (which also may be referred to as a “medical device” or simply an “implant”). Respective X, Y, and Z axes are shown in the figures to facilitate description of certain features of the illustrated embodiment and relationships therebetween. Certain similarities and differences between the implant 200 and the implant 100 described above will be appreciated from the drawings and the following description. Corresponding reference numbers are used for corresponding features, which generally may be configured in a manner similar to the features described above unless indicated otherwise. In some embodiments, the medical implant 200 may be a stent, such as an intravascular stent, although various other configurations of the implant 200 may be used in other embodiments. In some embodiments, the medical implant 200 may be formed as an elongated structure having a longitudinal axis A_{L} (extending in the Y direction). For example, when the implant 200 is a stent, the longitudinal axis A_{L} may be the axis with respect to which the stent radially expands. The implant 200 may include a structural member 210 (which also may be referred to as a “base member” or a “base”) and a radiopaque marker 230 (which also may be referred to as a “radiopaque element,” a “radiopaque member,” a “radiopaque marker insert,” a “radiopaque insert,” or a “marker insert”) that is fixedly connected to the structural member 210. In other words, after assembly, the radiopaque marker 230 is not intended to be removed from the structural member 210. In some embodiments, the structural member 210 may be a beam of the medical implant 200. For example, when the implant 200 is a stent, the structural member 210 may be a beam of the stent, such as a strut of the stent.

As shown, the structural member 210 may have a first surface 212, a second surface 214 disposed opposite the first surface 212 (in the Z direction), and an opening 220 (which also may be referred to as a “hole,” a “through-hole,” or an “aperture”) that extends from the first surface 212 to the second surface 214 (in the Z direction). The structural member 210 may have a wall thickness between the first surface 212 and the second surface 214 (in the Z direction). In some embodiments, as shown, the structural member 210 may have a constant wall thickness between the first surface 212 and the second surface 214. In some embodiments, as shown, the first surface 212 and the second surface 214 may be curved surfaces. For such embodiments, references herein to a feature (e.g., an axis, a surface, etc.) extending “perpendicular” to the first surface 212 and/or the second surface 214 mean that the feature extends perpendicular to a tangent of the curved surface at a location where the feature intersects the first surface 212 and/or the second surface 214. In some embodiments, as shown, the first surface 212 and the second surface 214 may be partial-cylindrical surfaces that define the longitudinal axis A_{L} of the implant 200. In some embodiments, the first surface 212 and the second surface 214 may be planar surfaces. In some embodiments, the second surface 214 may extend parallel, or substantially parallel, to the first surface 212. In some embodiments, the first surface 212 may be an external surface of the implant 200, and the second surface 214 may be an internal surface of the implant 200. For example, when the implant 200 is a stent, the first surface 212 may be a radially outer surface of the implant 200, and the second surface 214 may be a radially inner surface of the implant 200. The opening 220 may have a central axis A_{C} extending (in the Z direction) through a center of the opening 220. In some embodiments, as shown, the central axis A_{C} may extend perpendicular, or substantially perpendicular, to the longitudinal axis A_{L}. In some embodiments, the central axis A_{C} may extend perpendicular, or substantially perpendicular, to one or both of the first surface 212 and the second surface 214. In some embodiments, when the implant 200 is a stent, the opening 220 may be an eyelet of the stent. In some embodiments, as shown in FIG. 2H, the radiopaque marker 230 may fill the opening 220. In some embodiments, the radiopaque marker 230 may be flush with each of the first surface 212 and the second surface 214, as shown.

The opening 220 may be defined by a plurality of surfaces and/or edges. According to the illustrated embodiment, the opening 220 may be defined by a first partial-cylindrical surface 222 (which also may be referred to as a “first curved surface”) and a second partial-cylindrical surface 224 (which also may be referred to as a “second curved surface”). As shown, the first partial-cylindrical surface 222 may define a first central axis A_{C1}, and the second partial-cylindrical surface 224 may define a second central axis A_{C2}.

As shown in FIG. 2F, the second central axis A_{C2} may be angled (in the X-Z direction) relative to the first central axis A_{C1}. Further, the first central axis A_{C1} may be angled (in the X-Z direction) relative to the central axis A_{C}, and the second central axis A_{C2} may be angled (in the X-Z direction) relative to the central axis A_{C}. As shown, the first central axis A_{C1} may be angled relative to the central axis A_{C} in a first angular direction (the “+” X-Z direction in the illustrated embodiment), and the second central axis A_{C2} may be angled relative to the central axis A_{C} in a second angular direction (the “−”X-Z direction in the illustrated embodiment) that is opposite the first angular direction. In some embodiments, the second central axis A_{C2} and the first central axis A_{C1} may define a first acute angle α1 therebetween. In some embodiments, the first acute angle α1 may be within a range from 1 degree to 89 degrees. In some embodiments, the first acute angle α1 may be 20 degrees. In some embodiments, the first central axis A_{C1} and the central axis A_{C} may define a second acute angle α2 therebetween, and the second central axis A_{C2} and the central axis A_{C} may define a third acute angle α3 therebetween. In some embodiments, the second acute angle α2 may be within a range from 1 degree to 45 degrees, and the third acute angle α3 may be within a range from 1 degree to 45 degrees. In some embodiments, the second acute angle α2 may be equal to the third acute angle α3. In some embodiments, the second acute angle α2 may be 10 degrees, and the third acute angle α3 may be 10 degrees. As shown, the first acute angle α1 may be equal to a sum of the second acute angle α2 and the third acute angle α3.

As shown, each of the partial-cylindrical surfaces 222, 224 may have a constant radius of curvature. The first partial-cylindrical surface 222 may have a first radius of curvature, and the second partial-cylindrical surface 224 may have a second radius of curvature. In some embodiments, as shown, the second radius of curvature may be equal to the first radius of curvature. In some embodiments, the first partial-cylindrical surface 222 may extend from the first surface 212 to the second surface 214. In some embodiments, the second partial-cylindrical surface 224 may extend from the first surface 212 to the second surface 214.

As shown in FIG. 2E, the second central axis A_{C2} may be offset (in the Y direction) from the first central axis A_{C1} by a first distance D₁. In some embodiments, the first distance D₁ may be within a range from 40% to 90% of the first radius of curvature. In some embodiments, the first distance D₁ may be 66% of the first radius of curvature. In some embodiments, the first central axis A_{C1} may be offset from the second central axis A_{C2} by the first distance D₁ in a direction (the Y direction in the illustrated embodiment) extending perpendicular, or substantially perpendicular, to the central axis A_{C}. In some embodiments, the first central axis A_{C1} may be offset (in the Y direction) from the central axis A_{C} by a second distance D₂, and the second central axis A_{C2} may be offset (in the Y direction) from the central axis A_{C} by a third distance D₃. In some embodiments, the first central axis A_{C1} may be offset from the central axis A_{C} by the second distance D₂ in a first linear direction (the “+” Y direction in the illustrated embodiment), and the second central axis A_{C2} may be offset from the central axis A_{C} by the third distance D₃ in a second linear direction (the “−” Y direction in the illustrated embodiment) that is opposite the first linear direction. In some embodiments, the second distance D₂ may be within a range from 20% to 45% of the first radius of curvature, and the third distance D₃ may be within a range from 20% to 45% of the first radius of curvature. In some embodiments, the second distance D₂ may be equal to the third distance D₃.

In some embodiments, the opening 220 may be further defined by edges formed along intersections between the partial-cylindrical surfaces 222, 224. As shown, the opening 220 may be further defined by a first edge disposed along a first intersection between the second partial-cylindrical surface 224 and the first partial-cylindrical surface 222, and a second edge disposed along an intersection between the second partial-cylindrical surface 224 and the first partial-cylindrical surface 222. As shown, each of the edges may have a contoured shape corresponding to the angular and offset relationships of the central axes A_{C1}, A_{C2} and the radii of curvature of the partial-cylindrical surfaces 222, 224. In some embodiments, the opening 220 may be defined entirely by the first partial-cylindrical surface 222, the second partial-cylindrical surface 224, the first edge, and the second edge.

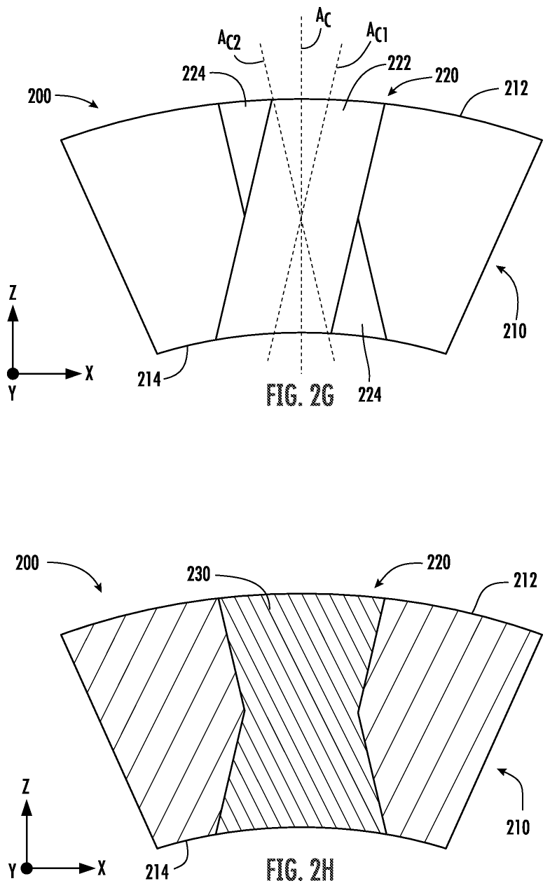

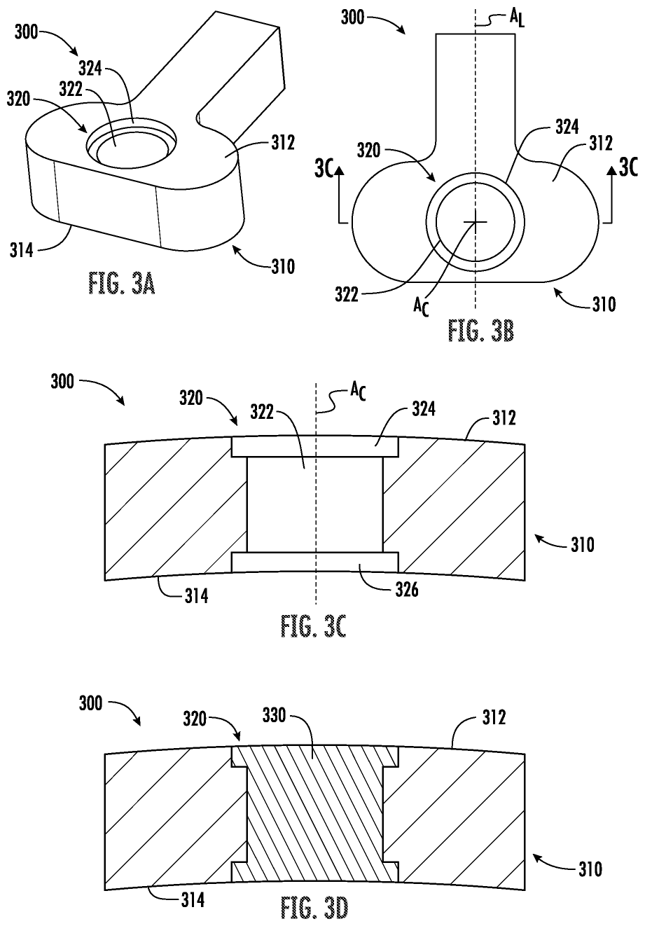

FIGS. 3A-3D depict a portion of another example medical implant 300 (which also may be referred to as a “medical device” or simply an “implant”). Certain similarities and differences between the implant 300 and the implants 100, 200 described above will be appreciated from the drawings and the following description. Corresponding reference numbers are used for corresponding features, which generally may be configured in a manner similar to the features described above unless indicated otherwise. In some embodiments, the medical implant 300 may be a stent, such as an intravascular stent, although various other configurations of the implant 300 may be used in other embodiments. In some embodiments, the medical implant 300 may be formed as an elongated structure having a longitudinal axis A_{L}. For example, when the implant 300 is a stent, the longitudinal axis A_{L} may be the axis with respect to which the stent radially expands. The implant 300 may include a structural member 310 (which also may be referred to as a “base member” or a “base”) and a radiopaque marker 330 (which also may be referred to as a “radiopaque element,” a “radiopaque member,” a “radiopaque marker insert,” a “radiopaque insert,” or a “marker insert”) that is fixedly connected to the structural member 310. In other words, after assembly, the radiopaque marker 330 is not intended to be removed from the structural member 310. In some embodiments, the structural member 310 may be a beam of the medical implant 300. For example, when the implant 300 is a stent, the structural member 310 may be a beam of the stent, such as a strut of the stent.

As shown, the structural member 310 may have a first surface 312, a second surface 314 disposed opposite the first surface 312, and an opening 320 (which also may be referred to as a “hole,” a “through-hole,” or an “aperture”) that extends from the first surface 312 to the second surface 314. The structural member 310 may have a wall thickness between the first surface 312 and the second surface 314. In some embodiments, as shown, the structural member 310 may have a constant wall thickness between the first surface 312 and the second surface 314. In some embodiments, as shown, the first surface 312 and the second surface 314 may be curved surfaces. For such embodiments, references herein to a feature (e.g., an axis, a surface, etc.) extending “perpendicular” to the first surface 312 and/or the second surface 314 mean that the feature extends perpendicular to a tangent of the curved surface at a location where the feature intersects the first surface 312 and/or the second surface 314. In some embodiments, as shown, the first surface 312 and the second surface 314 may be partial-cylindrical surfaces that define the longitudinal axis A_{L} of the implant 300. In some embodiments, the first surface 312 and the second surface 314 may be planar surfaces. In some embodiments, the second surface 314 may extend parallel, or substantially parallel, to the first surface 312. In some embodiments, the first surface 312 may be an external surface of the implant 300, and the second surface 314 may be an internal surface of the implant 300. For example, when the implant 300 is a stent, the first surface 312 may be a radially outer surface of the implant 300, and the second surface 314 may be a radially inner surface of the implant 300. The opening 320 may have a central axis A_{C} extending through a center of the opening 320. In some embodiments, as shown, the central axis A_{C} may extend perpendicular, or substantially perpendicular, to the longitudinal axis A_{L}. In some embodiments, the central axis A_{C} may extend perpendicular, or substantially perpendicular, to one or both of the first surface 312 and the second surface 314. In some embodiments, when the implant 300 is a stent, the opening 320 may be an eyelet of the stent. In some embodiments, as shown in FIG. 3D, the radiopaque marker 330 may fill the opening 320. In some embodiments, the radiopaque marker 330 may be flush with each of the first surface 312 and the second surface 314, as shown.

The opening 320 may be defined by a plurality of surfaces and/or edges. According to the illustrated embodiment, the opening 320 may be defined by a cylindrical surface 322, a first counterbore 324 extending from the first surface 312 to the cylindrical surface 322, and a second counterbore 326 extending from the second surface 314 to the cylindrical surface 322. In some embodiments, the cylindrical surface 322 may have a first diameter, the first counterbore 324 may have a second diameter that is within a range of 105% to 130% of the first diameter, and the second counterbore 326 may have a third diameter that is within a range of 105% to 130% of the first diameter. In some embodiments, as shown, the third diameter may be equal to the second diameter. In some embodiments, the first counterbore 324 may have a first depth relative to the first surface 312 that is within a range of 5% to 40% of the wall thickness between the first surface 312 and the second surface 314, and the second counterbore 326 may have a second depth relative to the second surface 314 that is within a range of 5% to 40% of the wall thickness between the first surface 312 and the second surface 314.

In some embodiments, the opening 320 may be further defined by edges formed by the counterbores 324, 326 and along intersections between the cylindrical surface 322 and the counterbores 324, 326. As shown, the opening 320 may be further defined by a first edge formed by the first counterbore 324, a second edge formed by the second counterbore 326, a third edge disposed along a first intersection between the first counterbore 324 and the cylindrical surface 322, and a fourth edge disposed along a second intersection between the second counterbore 326 and the cylindrical surface 322. As shown, each of the edges may have an annular shape. In some embodiments, the opening 320 may be defined entirely by the cylindrical surface 322, the first counterbore 324, the second counterbore 326, the first edge, the second edge, the third edge, and the fourth edge.

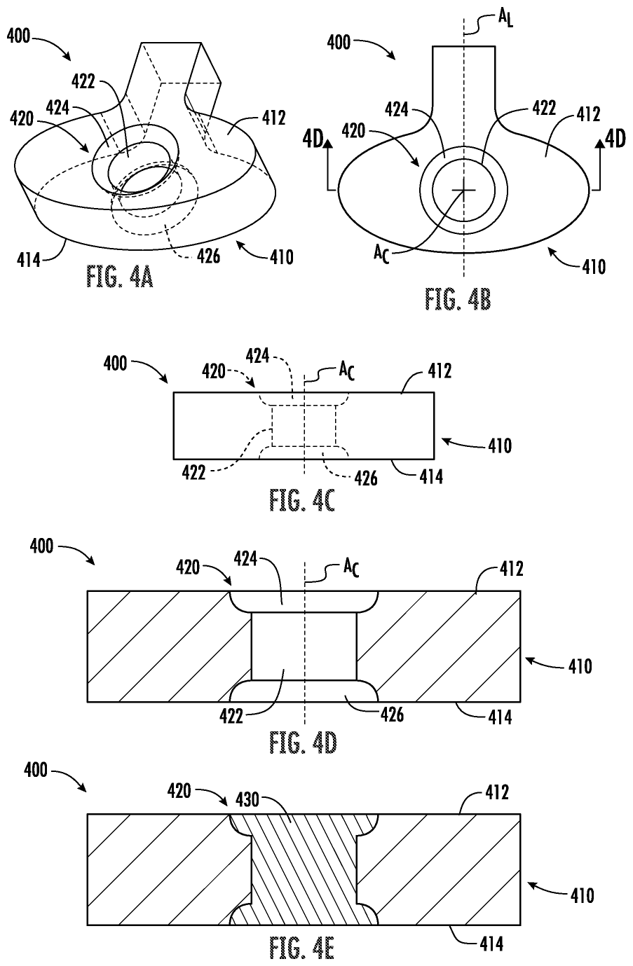

FIGS. 4A-4E depict a portion of another example medical implant 400 (which also may be referred to as a “medical device” or simply an “implant”). Certain similarities and differences between the implant 400 and the implants 100, 200, 300 described above will be appreciated from the drawings and the following description. Corresponding reference numbers are used for corresponding features, which generally may be configured in a manner similar to the features described above unless indicated otherwise. In some embodiments, the medical implant 400 may be a stent, such as an intravascular stent, although various other configurations of the implant 400 may be used in other embodiments. In some embodiments, the medical implant 400 may be formed as an elongated structure having a longitudinal axis A_{L}. For example, when the implant 400 is a stent, the longitudinal axis A_{L} may be the axis with respect to which the stent radially expands. The implant 400 may include a structural member 410 (which also may be referred to as a “base member” or a “base”) and a radiopaque marker 430 (which also may be referred to as a “radiopaque element,” a “radiopaque member,” a “radiopaque marker insert,” a “radiopaque insert,” or a “marker insert”) that is fixedly connected to the structural member 410. In other words, after assembly, the radiopaque marker 430 is not intended to be removed from the structural member 410. In some embodiments, the structural member 410 may be a beam of the medical implant 400. For example, when the implant 400 is a stent, the structural member 410 may be a beam of the stent, such as a strut of the stent.

As shown, the structural member 410 may have a first surface 412, a second surface 414 disposed opposite the first surface 412, and an opening 420 (which also may be referred to as a “hole,” a “through-hole,” or an “aperture”) that extends from the first surface 412 to the second surface 414. The structural member 410 may have a wall thickness between the first surface 412 and the second surface 414. In some embodiments, as shown, the structural member 410 may have a constant wall thickness between the first surface 412 and the second surface 414. In some embodiments, the first surface 412 and the second surface 414 may be curved surfaces. For such embodiments, references herein to a feature (e.g., an axis, a surface, etc.) extending “perpendicular” to the first surface 412 and/or the second surface 414 mean that the feature extends perpendicular to a tangent of the curved surface at a location where the feature intersects the first surface 412 and/or the second surface 414. In some embodiments, the first surface 412 and the second surface 414 may be partial-cylindrical surfaces that define the longitudinal axis A_{L} of the implant 400. In some embodiments, as shown, the first surface 412 and the second surface 414 may be planar surfaces. In some embodiments, the second surface 414 may extend parallel, or substantially parallel, to the first surface 412. In some embodiments, the first surface 412 may be an external surface of the implant 400, and the second surface 414 may be an internal surface of the implant 400. For example, when the implant 400 is a stent, the first surface 412 may be a radially outer surface of the implant 400, and the second surface 414 may be a radially inner surface of the implant 400. The opening 420 may have a central axis A_{C} extending through a center of the opening 420. In some embodiments, as shown, the central axis A_{C} may extend perpendicular, or substantially perpendicular, to the longitudinal axis A_{L}. In some embodiments, the central axis A_{C} may extend perpendicular, or substantially perpendicular, to one or both of the first surface 412 and the second surface 414. In some embodiments, when the implant 400 is a stent, the opening 420 may be an eyelet of the stent. In some embodiments, as shown in FIG. 4E, the radiopaque marker 430 may fill the opening 420. In some embodiments, the radiopaque marker 430 may be flush with each of the first surface 412 and the second surface 414, as shown.

The opening 420 may be defined by a plurality of surfaces and/or edges. According to the illustrated embodiment, the opening 420 may be defined by a cylindrical surface 422 (which also may be referred to as a “first curved surface”), a first concave surface 424 (which also may be referred to as a “second curved surface”) having an annular shape and extending from the first surface 412 to the cylindrical surface 422, and a second concave surface 426 (which also may be referred to as a “third curved surface”) having an annular shape and extending from the second surface 414 to the cylindrical surface 422. In some embodiments, the cylindrical surface 422 may have a first diameter, the first concave surface 424 may have a first minimum diameter that is equal to the first diameter and a first maximum diameter that is within a range of 105% to 130% of the first diameter, and the second concave surface 426 may have a second minimum diameter that is equal to the first diameter and a second maximum diameter that is within a range of 105% to 130% of the first diameter. In some embodiments, as shown, the first maximum diameter may be equal to the second maximum diameter. In some embodiments, the first concave surface 424 may have a first depth relative to the first surface 412 that is within a range of 5% to 40% of the wall thickness between the first surface 412 and the second surface 414, and the second concave surface 426 may have a second depth relative to the second surface 414 that is within a range of 5% to 40% of the wall thickness between the first surface 412 and the second surface 414.

In some embodiments, the opening 420 may be further defined by edges formed along intersections between the cylindrical surface 422 and the concave surfaces 424, 426. As shown, the opening 420 may be further defined by a first edge disposed along a first intersection between the first concave surface 424 and the cylindrical surface 422, and a second edge disposed along a second intersection between the second concave surface 426 and the cylindrical surface 422. As shown, each of the edges may have an annular shape. In some embodiments, the opening 420 may be defined entirely by the cylindrical surface 422, the first concave surface 424, the second concave surface 426, the first edge, and the second edge.

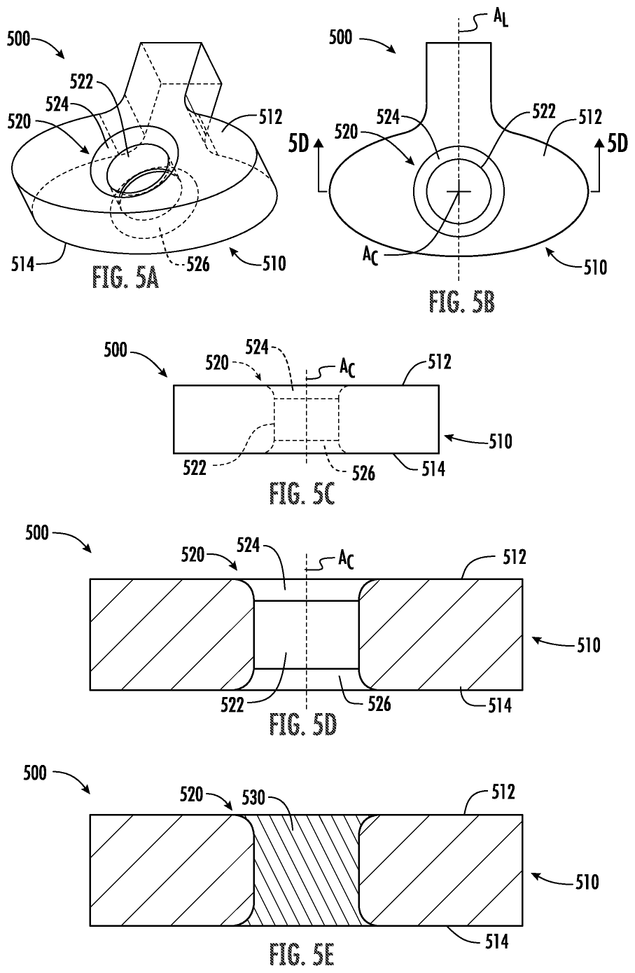

FIGS. 5A-5E depict a portion of another example medical implant 500 (which also may be referred to as a “medical device” or simply an “implant”). Certain similarities and differences between the implant 500 and the implants 100, 200, 300, 400 described above will be appreciated from the drawings and the following description. Corresponding reference numbers are used for corresponding features, which generally may be configured in a manner similar to the features described above unless indicated otherwise. In some embodiments, the medical implant 500 may be a stent, such as an intravascular stent, although various other configurations of the implant 500 may be used in other embodiments. In some embodiments, the medical implant 500 may be formed as an elongated structure having a longitudinal axis A_{L}. For example, when the implant 500 is a stent, the longitudinal axis A_{L} may be the axis with respect to which the stent radially expands. The implant 500 may include a structural member 510 (which also may be referred to as a “base member” or a “base”) and a radiopaque marker 530 (which also may be referred to as a “radiopaque element,” a “radiopaque member,” a “radiopaque marker insert,” a “radiopaque insert,” or a “marker insert”) that is fixedly connected to the structural member 510. In other words, after assembly, the radiopaque marker 530 is not intended to be removed from the structural member 510. In some embodiments, the structural member 510 may be a beam of the medical implant 500. For example, when the implant 500 is a stent, the structural member 510 may be a beam of the stent, such as a strut of the stent.

As shown, the structural member 510 may have a first surface 512, a second surface 514 disposed opposite the first surface 512, and an opening 520 (which also may be referred to as a “hole,” a “through-hole,” or an “aperture”) that extends from the first surface 512 to the second surface 514. The structural member 510 may have a wall thickness between the first surface 512 and the second surface 514. In some embodiments, as shown, the structural member 510 may have a constant wall thickness between the first surface 512 and the second surface 514. In some embodiments, the first surface 512 and the second surface 514 may be curved surfaces. For such embodiments, references herein to a feature (e.g., an axis, a surface, etc.) extending “perpendicular” to the first surface 512 and/or the second surface 514 mean that the feature extends perpendicular to a tangent of the curved surface at a location where the feature intersects the first surface 512 and/or the second surface 514. In some embodiments, the first surface 512 and the second surface 514 may be partial-cylindrical surfaces that define the longitudinal axis A_{L} of the implant 500. In some embodiments, as shown, the first surface 512 and the second surface 514 may be planar surfaces. In some embodiments, the second surface 514 may extend parallel, or substantially parallel, to the first surface 512. In some embodiments, the first surface 512 may be an external surface of the implant 500, and the second surface 514 may be an internal surface of the implant 500. For example, when the implant 500 is a stent, the first surface 512 may be a radially outer surface of the implant 500, and the second surface 514 may be a radially inner surface of the implant 500. The opening 520 may have a central axis A_{C} extending through a center of the opening 520. In some embodiments, as shown, the central axis A_{C} may extend perpendicular, or substantially perpendicular, to the longitudinal axis A_{L}. In some embodiments, the central axis A_{C} may extend perpendicular, or substantially perpendicular, to one or both of the first surface 512 and the second surface 514. In some embodiments, when the implant 500 is a stent, the opening 520 may be an eyelet of the stent. In some embodiments, as shown in FIG. 5E, the radiopaque marker 530 may fill the opening 520. In some embodiments, the radiopaque marker 530 may be flush with each of the first surface 512 and the second surface 514, as shown.

The opening 520 may be defined by a plurality of surfaces and/or edges. According to the illustrated embodiment, the opening 520 may be defined by a cylindrical surface 522 (which also may be referred to as a “first curved surface”), a first convex surface 524 (which also may be referred to as a “second curved surface”) having an annular shape and extending from the first surface 512 to the cylindrical surface 522, and a second convex surface 526 (which also may be referred to as a “third curved surface”) having an annular shape and extending from the second surface 514 to the cylindrical surface 522. In some embodiments, the cylindrical surface 522 may have a first diameter, the first convex surface 524 may have a first minimum diameter that is equal to the first diameter and a first maximum diameter that is within a range of 105% to 130% of the first diameter, and the second convex surface 526 may have a second minimum diameter that is equal to the first diameter and a second maximum diameter that is within a range of 105% to 130% of the first diameter. In some embodiments, as shown, the first maximum diameter may be equal to the second maximum diameter. In some embodiments, the first convex surface 524 may have a first depth relative to the first surface 512 that is within a range of 5% to 40% of the wall thickness between the first surface 512 and the second surface 514, and the second convex surface 526 may have a second depth relative to the second surface 514 that is within a range of 5% to 40% of the wall thickness between the first surface 512 and the second surface 514.

In some embodiments, the opening 520 may be further defined by edges formed along intersections between the cylindrical surface 522 and the convex surfaces 524, 526. As shown, the opening 520 may be further defined by a first edge disposed along a first intersection between the first convex surface 524 and the cylindrical surface 522, and a second edge disposed along a second intersection between the second convex surface 526 and the cylindrical surface 522. As shown, each of the edges may have an annular shape. In some embodiments, the opening 520 may be defined entirely by the cylindrical surface 522, the first convex surface 524, the second convex surface 526, the first edge, and the second edge.

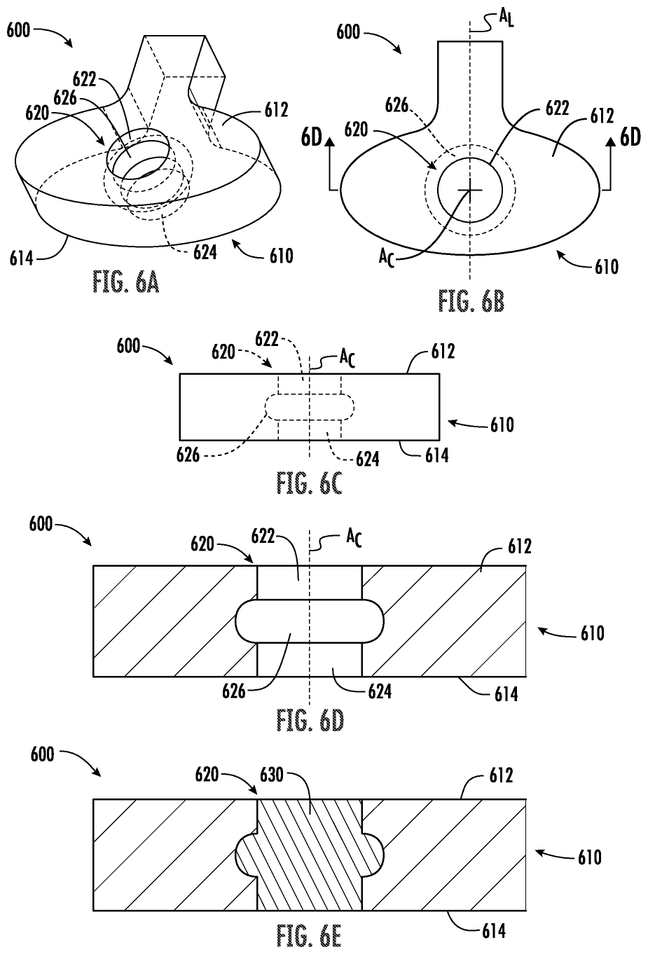

FIGS. 6A-6E depict a portion of another example medical implant 600 (which also may be referred to as a “medical device” or simply an “implant”). Certain similarities and differences between the implant 600 and the implants 100, 200, 300, 400, 500 described above will be appreciated from the drawings and the following description. Corresponding reference numbers are used for corresponding features, which generally may be configured in a manner similar to the features described above unless indicated otherwise. In some embodiments, the medical implant 600 may be a stent, such as an intravascular stent, although various other configurations of the implant 600 may be used in other embodiments. In some embodiments, the medical implant 600 may be formed as an elongated structure having a longitudinal axis A_{L}. For example, when the implant 600 is a stent, the longitudinal axis A_{L} may be the axis with respect to which the stent radially expands. The implant 600 may include a structural member 610 (which also may be referred to as a “base member” or a “base”) and a radiopaque marker 630 (which also may be referred to as a “radiopaque element,” a “radiopaque member,” a “radiopaque marker insert,” a “radiopaque insert,” or a “marker insert”) that is fixedly connected to the structural member 610. In other words, after assembly, the radiopaque marker 630 is not intended to be removed from the structural member 610. In some embodiments, the structural member 610 may be a beam of the medical implant 600. For example, when the implant 600 is a stent, the structural member 610 may be a beam of the stent, such as a strut of the stent.

As shown, the structural member 610 may have a first surface 612, a second surface 614 disposed opposite the first surface 612, and an opening 620 (which also may be referred to as a “hole,” a “through-hole,” or an “aperture”) that extends from the first surface 612 to the second surface 614. The structural member 610 may have a wall thickness between the first surface 612 and the second surface 614. In some embodiments, as shown, the structural member 610 may have a constant wall thickness between the first surface 612 and the second surface 614. In some embodiments, the first surface 612 and the second surface 614 may be curved surfaces. For such embodiments, references herein to a feature (e.g., an axis, a surface, etc.) extending “perpendicular” to the first surface 612 and/or the second surface 614 mean that the feature extends perpendicular to a tangent of the curved surface at a location where the feature intersects the first surface 612 and/or the second surface 614. In some embodiments, the first surface 612 and the second surface 614 may be partial-cylindrical surfaces that define the longitudinal axis A_{L} of the implant 600. In some embodiments, as shown, the first surface 612 and the second surface 614 may be planar surfaces. In some embodiments, the second surface 614 may extend parallel, or substantially parallel, to the first surface 612. In some embodiments, the first surface 612 may be an external surface of the implant 600, and the second surface 614 may be an internal surface of the implant 600. For example, when the implant 600 is a stent, the first surface 612 may be a radially outer surface of the implant 600, and the second surface 614 may be a radially inner surface of the implant 600. The opening 620 may have a central axis A_{C} extending through a center of the opening 620. In some embodiments, as shown, the central axis A_{C} may extend perpendicular, or substantially perpendicular, to the longitudinal axis A_{L}. In some embodiments, the central axis A_{C} may extend perpendicular, or substantially perpendicular, to one or both of the first surface 612 and the second surface 614. In some embodiments, when the implant 600 is a stent, the opening 620 may be an eyelet of the stent. In some embodiments, as shown in FIG. 6E, the radiopaque marker 630 may fill the opening 620. In some embodiments, the radiopaque marker 630 may be flush with each of the first surface 612 and the second surface 614, as shown.

The opening 620 may be defined by a plurality of surfaces and/or edges. According to the illustrated embodiment, the opening 620 may be defined by a first cylindrical surface 622 (which also may be referred to as a “first curved surface”) extending from the first surface 612 toward the second surface 614, a second cylindrical surface 624 (which also may be referred to as a “second curved surface”) extending from the second surface 614 toward the first surface 612, and an annular groove 626 (which also may be referred to as a “third curved surface”) disposed between the first cylindrical surface 622 and the second cylindrical surface 624. In some embodiments, the first cylindrical surface 622 may have a first diameter, the second cylindrical surface 624 may have a second diameter that is equal to the first diameter, and the annular groove 626 may have a minimum diameter that is equal to the first diameter and a maximum diameter that is within a range of 105% to 130% of the first diameter. In some embodiments, the annular groove 626 may have a thickness in the direction between the first surface 612 and the second surface 614 that is within a range of 30% to 70% of the wall thickness between the first surface 612 and the second surface 614.

In some embodiments, the opening 620 may be further defined by edges formed along intersections between the annular groove 626 and the cylindrical surfaces 622, 624. As shown, the opening 620 may be further defined by a first edge disposed along a first intersection between the annular groove 626 and the first cylindrical surface 622, and a second edge disposed along a second intersection between the annular groove 626 and the second cylindrical surface 624. As shown, each of the edges may have an annular shape. In some embodiments, the opening 620 may be defined entirely by the first cylindrical surface 622, the second cylindrical surface 624, the annular groove 626, the first edge, and the second edge.

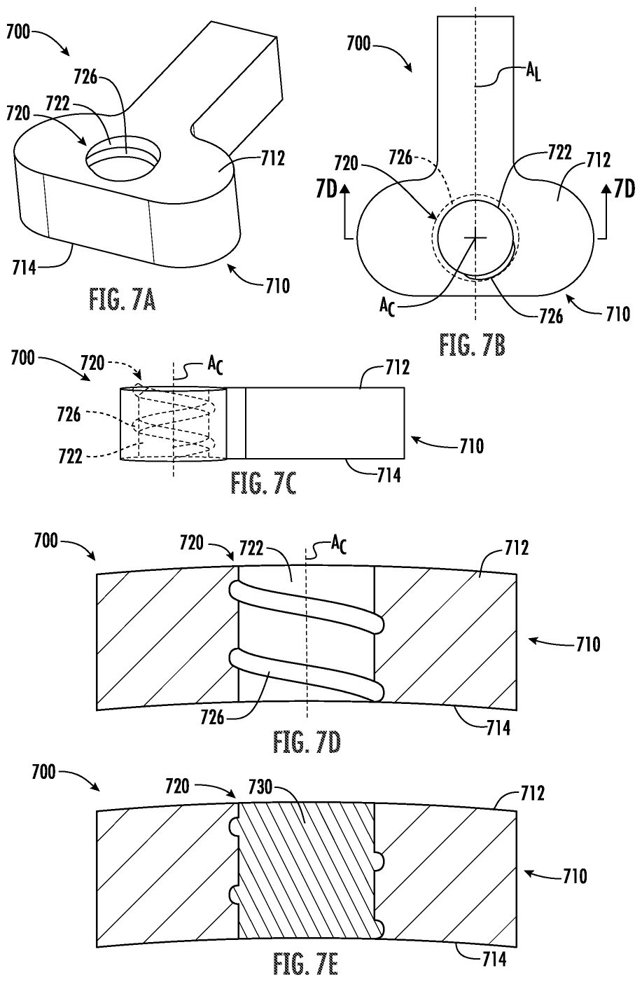

FIGS. 7A-7E depict a portion of another example medical implant 700 (which also may be referred to as a “medical device” or simply an “implant”). Certain similarities and differences between the implant 700 and the implants 100, 200, 300, 400, 500, 600 described above will be appreciated from the drawings and the following description. Corresponding reference numbers are used for corresponding features, which generally may be configured in a manner similar to the features described above unless indicated otherwise. In some embodiments, the medical implant 700 may be a stent, such as an intravascular stent, although various other configurations of the implant 700 may be used in other embodiments. In some embodiments, the medical implant 700 may be formed as an elongated structure having a longitudinal axis A_{L}. For example, when the implant 700 is a stent, the longitudinal axis A_{L} may be the axis with respect to which the stent radially expands. The implant 700 may include a structural member 710 (which also may be referred to as a “base member” or a “base”) and a radiopaque marker 730 (which also may be referred to as a “radiopaque element,” a “radiopaque member,” a “radiopaque marker insert,” a “radiopaque insert,” or a “marker insert”) that is fixedly connected to the structural member 710. In other words, after assembly, the radiopaque marker 730 is not intended to be removed from the structural member 710. In some embodiments, the structural member 710 may be a beam of the medical implant 700. For example, when the implant 700 is a stent, the structural member 710 may be a beam of the stent, such as a strut of the stent.

As shown, the structural member 710 may have a first surface 712, a second surface 714 disposed opposite the first surface 712, and an opening 720 (which also may be referred to as a “hole,” a “through-hole,” or an “aperture”) that extends from the first surface 712 to the second surface 714. The structural member 710 may have a wall thickness between the first surface 712 and the second surface 714. In some embodiments, as shown, the structural member 710 may have a constant wall thickness between the first surface 712 and the second surface 714. In some embodiments, as shown, the first surface 712 and the second surface 714 may be curved surfaces. For such embodiments, references herein to a feature (e.g., an axis, a surface, etc.) extending “perpendicular” to the first surface 712 and/or the second surface 714 mean that the feature extends perpendicular to a tangent of the curved surface at a location where the feature intersects the first surface 712 and/or the second surface 714. In some embodiments, as shown, the first surface 712 and the second surface 714 may be partial-cylindrical surfaces that define the longitudinal axis A_{L} of the implant 700. In some embodiments, the first surface 712 and the second surface 714 may be planar surfaces. In some embodiments, the second surface 714 may extend parallel, or substantially parallel, to the first surface 712. In some embodiments, the first surface 712 may be an external surface of the implant 700, and the second surface 714 may be an internal surface of the implant 700. For example, when the implant 700 is a stent, the first surface 712 may be a radially outer surface of the implant 700, and the second surface 714 may be a radially inner surface of the implant 700. The opening 720 may have a central axis A_{C} extending through a center of the opening 720. In some embodiments, as shown, the central axis A_{C} may extend perpendicular, or substantially perpendicular, to the longitudinal axis A_{L}. In some embodiments, the central axis A_{C} may extend perpendicular, or substantially perpendicular, to one or both of the first surface 712 and the second surface 714. In some embodiments, when the implant 700 is a stent, the opening 720 may be an eyelet of the stent. In some embodiments, as shown in FIG. 7E, the radiopaque marker 730 may fill the opening 720. In some embodiments, the radiopaque marker 730 may be flush with each of the first surface 712 and the second surface 714, as shown.

The opening 720 may be defined by a plurality of surfaces and/or edges. According to the illustrated embodiment, the opening 720 may be defined by a partial-cylindrical surface 722 (which also may be referred to as a “first curved surface”) extending between the first surface 712 and the second surface 714, and a helical groove 726 (which also may be referred to as a “second curved surface”) extending between the first surface 712 and the second surface 714. In some embodiments, the partial-cylindrical surface 722 may have a first diameter, and the helical groove 726 may have a minimum diameter that is equal to the first diameter and a maximum diameter that is within a range of 105% to 130% of the first diameter. In some embodiments, the partial-cylindrical surface 722 may extend from the first surface 712 to the second surface 714, and the helical groove 726 may extend from the first surface 712 to the second surface 714.

In some embodiments, the opening 720 may be further defined by edges formed along intersections between the helical groove 726 and the partial-cylindrical surface 722. As shown, the opening 720 may be further defined by a first edge disposed along a first intersection between the helical groove 726 and the partial-cylindrical surface 722, and a second edge disposed along a second intersection between the helical groove 726 and the partial-cylindrical surface 722. As shown, each of the edges may have a helical shape. In some embodiments, the opening 720 may be defined entirely by the partial-cylindrical surface 722, the helical groove 726, the first edge, and the second edge.

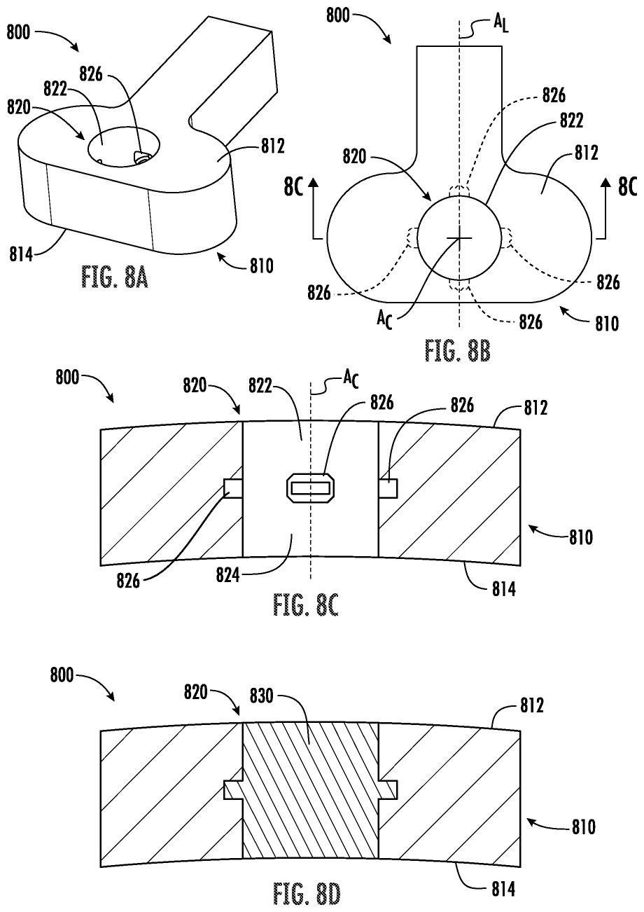

FIGS. 8A-8D depict a portion of another example medical implant 800 (which also may be referred to as a “medical device” or simply an “implant”). Certain similarities and differences between the implant 800 and the implants 100, 200, 300, 400, 500, 600, 700 described above will be appreciated from the drawings and the following description. Corresponding reference numbers are used for corresponding features, which generally may be configured in a manner similar to the features described above unless indicated otherwise. In some embodiments, the medical implant 800 may be a stent, such as an intravascular stent, although various other configurations of the implant 800 may be used in other embodiments. In some embodiments, the medical implant 800 may be formed as an elongated structure having a longitudinal axis A_{L}. For example, when the implant 800 is a stent, the longitudinal axis A_{L} may be the axis with respect to which the stent radially expands. The implant 800 may include a structural member 810 (which also may be referred to as a “base member” or a “base”) and a radiopaque marker 830 (which also may be referred to as a “radiopaque element,” a “radiopaque member,” a “radiopaque marker insert,” a “radiopaque insert,” or a “marker insert”) that is fixedly connected to the structural member 810. In other words, after assembly, the radiopaque marker 830 is not intended to be removed from the structural member 810. In some embodiments, the structural member 810 may be a beam of the medical implant 800. For example, when the implant 800 is a stent, the structural member 810 may be a beam of the stent, such as a strut of the stent.

As shown, the structural member 810 may have a first surface 812, a second surface 814 disposed opposite the first surface 812, and an opening 820 (which also may be referred to as a “hole,” a “through-hole,” or an “aperture”) that extends from the first surface 812 to the second surface 814. The structural member 810 may have a wall thickness between the first surface 812 and the second surface 814. In some embodiments, as shown, the structural member 810 may have a constant wall thickness between the first surface 812 and the second surface 814. In some embodiments, as shown, the first surface 812 and the second surface 814 may be curved surfaces. For such embodiments, references herein to a feature (e.g., an axis, a surface, etc.) extending “perpendicular” to the first surface 812 and/or the second surface 814 mean that the feature extends perpendicular to a tangent of the curved surface at a location where the feature intersects the first surface 812 and/or the second surface 814. In some embodiments, as shown, the first surface 812 and the second surface 814 may be partial-cylindrical surfaces that define the longitudinal axis A_{L} of the implant 800. In some embodiments, the first surface 812 and the second surface 814 may be planar surfaces. In some embodiments, the second surface 814 may extend parallel, or substantially parallel, to the first surface 812. In some embodiments, the first surface 812 may be an external surface of the implant 800, and the second surface 814 may be an internal surface of the implant 800. For example, when the implant 800 is a stent, the first surface 812 may be a radially outer surface of the implant 800, and the second surface 814 may be a radially inner surface of the implant 800. The opening 820 may have a central axis A_{C} extending through a center of the opening 820. In some embodiments, as shown, the central axis A_{C} may extend perpendicular, or substantially perpendicular, to the longitudinal axis A_{L}. In some embodiments, the central axis A_{C} may extend perpendicular, or substantially perpendicular, to one or both of the first surface 812 and the second surface 814. In some embodiments, when the implant 800 is a stent, the opening 820 may be an eyelet of the stent. In some embodiments, as shown in FIG. 8D, the radiopaque marker 830 may fill the opening 820. In some embodiments, the radiopaque marker 830 may be flush with each of the first surface 812 and the second surface 814, as shown.

The opening 820 may be defined by a plurality of surfaces and/or edges. According to the illustrated embodiment, the opening 820 may be defined by a first cylindrical surface 822 (which also may be referred to as a “first curved surface”) extending from the first surface 812 toward the second surface 814, a second cylindrical surface 824 (which also may be referred to as a “second curved surface”) extending from the second surface 814 toward the first surface 812, and a circumferential array of recesses 826 disposed around the central axis A_{C} and between the first cylindrical surface 822 and the second cylindrical surface 824. Although four (4) recesses 826 are shown in the illustrated embodiment, more or fewer recesses 826 may be used in other embodiments. In some embodiments, as shown, the recesses 826 may be equally spaced apart from one another in the circumferential direction around the central axis A_{C}. In some embodiments, the first cylindrical surface 822 may have a first diameter, the second cylindrical surface 824 may have a second diameter that is equal to the first diameter, open ends of the recesses 826 may define a minimum diameter that is equal to the first diameter, and closed ends of the recesses 826 may define a maximum diameter that is within a range of 105% to 130% of the first diameter. In some embodiments, each of the recesses 826 may have a thickness in the direction between the first surface 812 and the second surface 814 that is within a range of 10% to 50% of the wall thickness between the first surface 812 and the second surface 814.

In some embodiments, the opening 820 may be further defined by edges formed along intersections between the cylindrical surfaces 822, 824 and the open ends of the recesses 826. As shown, the opening 820 may be further defined by a first edge formed along the perimeter of the open end of the first recess 826, a second edge formed along the perimeter of the open end of the second recess 826, a third edge formed along the perimeter of the open end of the third recess 826, and a fourth edge formed along the perimeter of the open end of the fourth recess 826. As shown, each of the edges may have a shape corresponding to the shape of the open ends of the recesses 826. In some embodiments, the opening 820 may be defined entirely by the first cylindrical surface 822, the second cylindrical surface 824, the recesses 826, respective partial-cylindrical surfaces disposed between adjacent pairs of the recesses 826, the first edge, the second edge, the third edge, and the fourth edge.

Although specific embodiments of the disclosure have been described, one of ordinary skill in the art will recognize that numerous other modifications and alternative embodiments are within the scope of the disclosure. For example, while various illustrative implementations and structures have been described in accordance with embodiments of the disclosure, one of ordinary skill in the art will appreciate that numerous other modifications to the illustrative implementations and structures described herein are also within the scope of this disclosure.

Although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments do not include, certain features, elements, and/or steps. Thus, such conditional language is not generally intended to imply that features, elements, and/or steps are in any way required for one or more embodiments.

What is claimed is:

1. A medical implant comprising:
a structural member comprising a first surface, a second surface disposed opposite the first surface, and an opening extending from the first surface to the second surface, wherein the opening is defined by:
a first partial-cylindrical surface defining a first central axis; and
a second partial-cylindrical surface defining a second central axis that is angled relative to the first central axis; and
a radiopaque marker disposed within the opening.

2. The medical implant of claim 1, wherein the first surface and the second surface are curved surfaces.

3. The medical implant of claim 1, wherein the first surface and the second surface are partial-cylindrical surfaces defining a longitudinal axis of the medical implant.

4. The medical implant of claim 1, wherein the first surface and the second surface are planar surfaces, and wherein the second surface extends parallel to the first surface.

5. The medical implant of claim 1, wherein the first surface is an external surface of the medical implant, and wherein the second surface is an internal surface of the medical implant.

6. The medical implant of claim 1, wherein the second central axis and the first central axis define a first acute angle therebetween.

7. The medical implant of claim 6, wherein the opening defines a central axis that extends perpendicular to the first surface, wherein the first central axis is angled relative to the central axis of the opening, and wherein the second central axis is angled relative to the central axis of the opening.

8. The medical implant of claim 7, wherein the first central axis is angled relative to the central axis of the opening in a first angular direction, and wherein the second central axis is angled relative to the central axis of the opening in a second angular direction that is opposite the first angular direction.

9. The medical implant of claim 7, wherein the first central axis and the central axis of the opening define a second acute angle therebetween, and wherein the second central axis and the central axis of the opening define a third acute angle therebetween.

10. The medical implant of claim 9, wherein the second acute angle is equal to the third acute angle.

11. The medical implant of claim 7, wherein the central axis of the opening extends perpendicular to a longitudinal axis of the medical implant.

12. The medical implant of claim 7, wherein the first central axis is offset from the second central axis by a first distance.

13. The medical implant of claim 12, wherein the first partial-cylindrical surface has a first radius of curvature, and wherein the second partial-cylindrical surface has a second radius of curvature that is equal to the first radius of curvature.

14. The medical implant of claim 12, wherein the first central axis is offset from the second central axis by the first distance in a direction extending perpendicular to the central axis of the opening.

15. The medical implant of claim 12, wherein the first central axis is offset from the central axis of the opening by a second distance in a first linear direction, and wherein the second central axis is offset from the central axis of the opening by a third distance in a second linear direction that is opposite the first linear direction.

16. The medical implant of claim 15, wherein the second distance is equal to the third distance.

17. The medical implant of claim 1, wherein the first partial-cylindrical surface extends from the first surface to the second surface, and wherein the second partial-cylindrical surface extends from the first surface to the second surface.

18. The medical implant of claim 1, wherein the radiopaque marker fills the opening.

19. The medical implant of claim 1, wherein the medical implant is a stent.

20. The medical implant of claim 19, wherein the opening is an eyelet of the stent.
